(19) United States
(12) Patent Application Publication
(10) Pub. No.: US 2026/0108538 A1
(43) Pub. Date: Apr. 23, 2026

(54) CANNABINOID BASED NANOPLATFORM COMPOSITION AND METHODS FOR TREATING MENOPAUSE SYMPTOMS

(71) Applicant: Cannabis Bioscience International Holdings Inc, Houston, TX (US)

(72) Inventors: Rosangel del Valle Andrades Fuentes, Caracas (VE); Nancy Paola Duarte Delgado, Bogotá (CO); Nancy Rodrigues Das Fontes, Viseu (PT); Stephanie Dona Hartmann, Caracas (VE); Ana Maria Maldonado Vacca, Bogotá (CO); Maria Angelica Clavijo Romero, Santa Marta (CO)

(73) Assignee: Cannabis Bioscience International Holdings Inc, Houston, TX (US)

(21) Appl. No.: 18/921,140

(22) Filed: Oct. 21, 2024

(51) Int. Cl. A61K 31/00; A61K 9/02; A61K 9/48; A61K 31/05; A61K 31/352; A61K 36/48; A61K 47/14; A61K 47/22; A61K 47/44

(52) U.S. Cl. CPC A61K 31/658; A61K 9/02; A61K 9/4825; A61K 9/4858; A61K 31/05; A61K 31/352; A61K 36/48; A61K 47/14; A61K 47/22; A61K 47/44

(57) ABSTRACT

Aspects of disclosure relate to a composition and methodology for treating menopause symptoms utilizing a cannabinoid-based nanoplatform composition. The composition includes phytocannabinoids like CBD, CBG, and CBN, alongside isoflavones and polyphenols, all integrated into nanoplatform designed for enhanced bioavailability and controlled release. The formulation is designed to alleviate key menopause-related issues, including vasomotor symptoms (VMS), genitourinary syndrome of menopause (GSM), bone loss, mood disturbances, and sleep disorders, while addressing cardiovascular disease (CVD) risks. The composition combines cannabinoids, such as CBD, with isoflavones and polyphenols, and is incorporated into nanoplatforms for enhanced delivery and efficacy. Additionally, the disclosure includes a kit comprising oral capsules, intravaginal ovules, and instructions for use. The capsules contain a blend of cannabinoids, flavonoids, and polyphenols, while the ovules are composed of CBD and cocoa butter. The kit provides a comprehensive solution for managing menopause symptoms and mitigating cardiovascular risks.

### FIELD OF THE DISCLOSURE

The technical field of the disclosure relates to medicinal, therapeutic, and healing cannabis and cannabinoids, as well as pharmaceutical cannabis and cannabinoids. The present disclosure relates to therapeutic compositions for the management of menopausal symptoms, incorporating cannabinoids as active agents and designed for oral and intravaginal administration. These compositions utilize advanced nanoplatforms to enhance bioavailability and therapeutic efficacy. This disclosure specifically addresses relief of menopausal conditions, including vasomotor symptoms, genitourinary syndrome of menopause (GSM), mood disturbances, sleep disorders, and bone health, with potential benefits for cardiovascular health. By combining cannabinoids with bioactive compounds such as flavonoids and polyphenols, the compositions offer a comprehensive approach to improving quality of life throughout the menopausal transition.

### BACKGROUND INFORMATION

Menopause is defined as the permanent cessation of menstrual cycles due to the decline of ovarian function, occurring either naturally with age or because of medical interventions such as ovary removal (Davis et al. 2015). Although often viewed as a single moment coinciding with the end of ovarian oocyte production, the transition into menopause spans several years, marked by predictable changes in a women's menstrual cycle (Takahashi and Johnson 2015).

The perimenopausal phase, characterized by fluctuating hormone levels and the onset of initial symptoms, precedes the final menstrual period. Perimenopause concludes one year after the last period, at which point it is highly probable that menstrual cycles will cease entirely. Perimenopause is a transitional period characterized by three distinct phases. The early menopausal transition involves irregular menstrual cycles, while the late menopausal transition is marked by at least 60 consecutive days without menstruation. The final stage, early postmenopause, encompasses the first year following the last menstrual period (Davis et al. 2015).

The symptoms linked to hormonal changes during the menopausal transition include vasomotor symptoms (VMS) such as hot flashes, decreased body temperature, increased heart rate, vaginal dryness and urinary issues (now termed “genitourinary syndrome of menopause”), sexual dysfunction, as well as mood and sleep disturbances (Roberts and Hickey 2016). The impact of menopause on quality of life largely depends on the presence and severity of its symptoms. VMS, are among the most common, affecting up to 75% of women. Urogenital atrophy and its consequences, mood changes, and sleep disturbances are also frequent symptoms (Davis et al. 2015).

The decline in estrogen levels during menopause is associated with an increased risk of cardiovascular disease (CVD). Estrogens play a protective role in the cardiovascular system by regulating lipid metabolism and promoting healthy blood vessel function (Ryczkowska et al., 2023). Endothelial dysfunction, which involves impaired blood vessel dilation, is a recognized biomarker of aging and a strong predictor of cardiovascular events in menopausal women. Postmenopausal women, particularly those with estrogen deficiency, experience a more rapid decline in endothelial function compared to premenopausal women. This accelerated vascular aging is further influenced by hormonal changes, traditional cardiovascular risk factors such as central adiposity, hypertension, and dyslipidemia, as well as vasomotor symptoms (Moreau et al., 2012).

The age of natural menopause varies globally, typically occurring around 49 years of age. Menopausal symptoms are experienced universally, but their specific manifestations differ across ethnicities. Women of European and Latin American descent frequently report central nervous system-related symptoms, including hot flashes, sleep disturbances, mood swings, irritability, and decreased libido. In contrast, the prevalence of vasomotor symptoms is lower in India and Middle Eastern countries, such as the United Arab Emirates and Saudi Arabia, compared to Western nations. African American women experience more persistent vasomotor symptoms and have a higher risk of cardiovascular disease post-menopause than other ethnic groups (Davis et al. 2015).

The persistence of menopausal symptoms significantly impacts the quality of life, driving an increased demand for effective treatments. While hormone therapy (HT) has been the standard for treating menopausal symptoms it comes with significant risks. These include cardiovascular disease (CVD), venous thromboembolism (VTE), breast cancer, and cognitive decline, with complications varying depending on the therapy type, composition, and timing of administration. Selective Estrogen Receptor Modulators (SERMs) offer an alternative by mimicking estrogens effects in some tissues while blocking them in others, but these too have side effects, such as an increased risk of VTE and the potential to worsen hot flashes (Motlani et al, 2023).

There is a clear need for a safer, more effective treatment option that addresses these symptoms without exposing patients to these risks. Current therapies do not provide comprehensive symptom management, and safer alternatives are limited. The disclosure provides a cannabinoid-based composition aimed at mitigating menopausal symptoms through oral and intravaginal administration. By targeting the endocannabinoid system, cannabinoids regulate mood, pain, and thermoregulation, addressing VMS, GSM, sleep disturbances, and mood disorders. Additionally, flavonoids and polyphenols in the composition contribute antioxidant, bone-protective, and mild estrogenic effects, with polyphenols further supporting cardiovascular health. This unique combination provides a holistic approach to symptom management and presents a safer alternative to traditional therapies, specifically designed to improve well-being in menopausal women.

### SUMMARY OF THE DISCLOSURE

This disclosure presents a comprehensive approach to managing menopausal symptoms through three main embodiments: (1) a cannabinoid-based composition, (2) methods of treatment, (3) a method of manufacturing, a (4) a therapeutic kit.

Composition: The primary embodiment is a cannabinoid-based composition, optionally supplemented with isoflavones, flavonoids, and polyphenols, designed to alleviate key menopause-related symptoms such as vasomotor symptoms (VMS), genitourinary syndrome of menopause (GSM), bone loss, mood disturbances, and sleep disorders. The composition is tailored to reduce long-term health risks associated with menopause, including cardiovascular disease (CVD), by leveraging the therapeutic properties of cannabinoids in combination with isoflavones and polyphenols to support hormonal balance, enhance endothelial function, and provide comprehensive symptom relief.

Method of Treatment: This embodiment describes a therapeutic method particularly beneficial for perimenopausal, menopausal, and postmenopausal women. The treatment regimen includes oral and intravaginal administration of the composition, providing tailored dosage forms such as daily oral capsules and intravaginal ovules. This approach offers effective relief from menopausal symptoms, including hot flashes, vaginal dryness, burning, dyspareunia, osteopenia, mood swings, and sleep disorders. By targeting the endocannabinoid system and utilizing nanoplatforms for enhanced bioavailability, this treatment method optimizes symptom management and mitigates associated health risks, such as CVD.

Method of Manufacturing: The disclosure includes a method for producing the composition, which involves integrating cannabinoids, such as CBD, with isoflavones and polyphenols into nanoplatforms for improved therapeutic delivery. The manufacturing method ensures consistent formulation and optimized bioavailability for both oral and intravaginal delivery routes, enhancing the composition effectiveness and stability.

The disclosure also encompasses a kit designed for user convenience, containing: (a) daily oral capsules with cannabinoids, flavonoids, and polyphenols; (b) intravaginal ovules containing CBD and cocoa butter; and (c) instructions for use to manage menopausal symptoms and reduce cardiovascular risk. This kit is tailored to meet the needs of women across all stages of menopause, providing a practical solution for alleviating symptoms such as hot flashes, GSM, mood disturbances, and sleep disorders. In summary, this disclosure offers a novel, safe, and comprehensive strategy to improve the well-being of women throughout the menopausal transition by addressing both menopausal symptoms and associated health risks.

### BRIEF DESCRIPTION OF THE FIGURES

FIG. 1 illustrates the multifaceted mechanisms by which phytocannabinoids (CBD, CBG, CBN), flavonoids (isoflavones, quercetin, myricetin), and polyphenols (resveratrol) may alleviate various symptoms and pathophysiological processes associated with menopause. Top Center: CBD engages the 5-HT1A serotonin receptors, modulating serotonin signaling pathways that are crucial for mood and anxiety regulation. By acting as a partial agonist at these receptors, CBD may reduce anxiety and promote emotional stability, addressing the mood swings and sleep disorders commonly experienced during menopause. Top Right: During menopause, decreased estrogen levels result in heightened osteoclast activity, which accelerates bone resorption and leads to osteoporosis. CBD, along with myricetin and resveratrol, may inhibit osteoclast activity and reduce bone resorption by interacting with TRPV1 channels and other pathways related to bone metabolism. This mechanism suggests that these compounds can help preserve bone density and reduce the risk of fractures. Bottom Right: Menopause-related alterations in lipid metabolism, such as increased LDL and decreased HDL levels, elevate the risk of atherosclerotic plaque formation and cardiovascular disease. Resveratrol and quercetin exert lipid-lowering effects by preventing LDL oxidation, improving HDL function, and reducing oxidative stress and inflammation. These actions collectively inhibit the progression of atherosclerosis, providing cardiovascular protection during menopause. Bottom Left: CBD interacts with CB1 and CB2 cannabinoid receptors to regulate the release of cytokines and chemokines, potentially reducing inflammation in tissues affected by menopause-related changes, such as the vaginal area. By reducing local inflammation and promoting immune balance, CBD can help alleviate conditions like vaginal dryness and vulvovaginal atrophy, which increase susceptibility to infections, thereby improving overall genitourinary health. Top Left: The loss of estrogen during menopause destabilizes the hypothalamic thermoregulatory center, leading to hot flashes and other vasomotor symptoms. Isoflavones (a class of flavonoids) mimic the thermoregulatory actions of estrogens on specific 5-HT1A and TRPV1 receptors, potentially restoring balance and alleviating hot flashes.

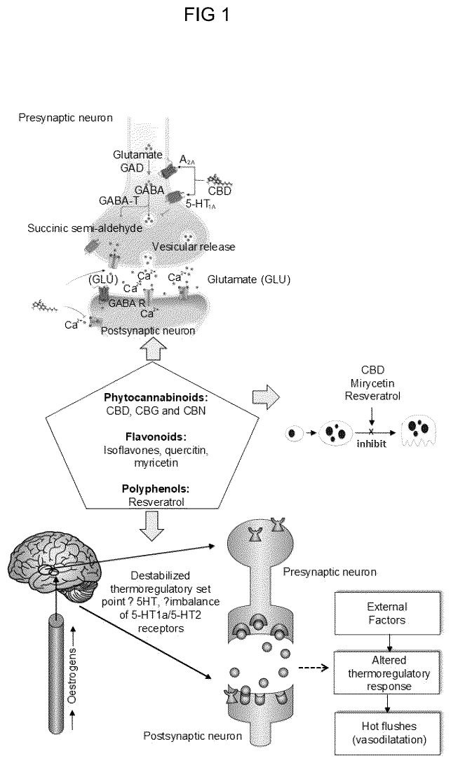

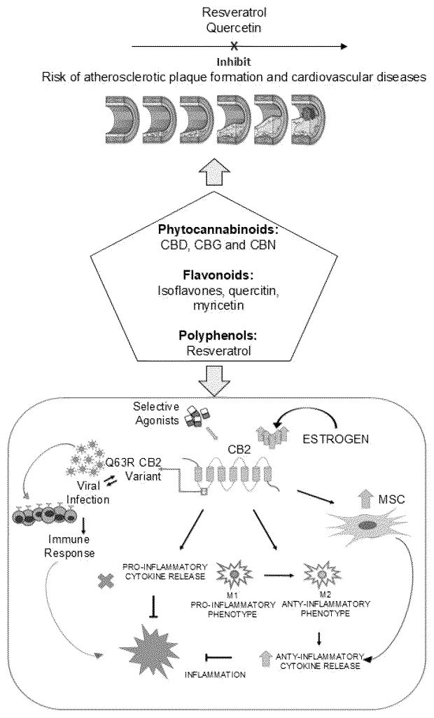

### DETAILED DESCRIPTION

### Definitions

During menopause, the decline in estrogen levels has far-reaching effects, not just on the reproductive system, but also on brain signaling. Estrogen plays a critical role in maintaining communication between the ovaries and the brain, particularly in areas associated with cognition, memory, and emotional regulation. The hormone exerts neuroprotective effects by modulating neurotransmitters, supporting synaptic plasticity, and regulating blood flow within the brain. As estrogen levels decrease during menopause, this communication is disrupted, which can lead to cognitive dysfunction. Estrogen normally enhances the function of neurotransmitters like serotonin, acetylcholine, and dopamine, all of which are essential for mood regulation, learning, and memory processes. Without sufficient estrogen, these pathways become less efficient, contributing to symptoms such as memory lapses, difficulty concentrating, and slower information processing, commonly referred to as “menopause brain” or “brain fog.

Estrogen deficiency during menopause signals to the body that the reproductive phase has ended, leading to changes in various systems. Estrogen, which supports bone health, vaginal lubrication, skin elasticity, and fat distribution, declines, causing the body to shift resources away from maintaining reproductive tissues. This results in weaker bones, vaginal dryness, skin thinning, and changes in metabolism, as the body no longer prioritizes fertility. The hormonal shift is a natural biological response indicating that the body reproductive function has been fulfilled.

Ovarian insufficiency and the subsequent decrease in estrogen levels during menopause are associated with vasomotor/genitourinary symptoms, weight gain, cardiometabolic disease, and bone loss (Wright, 2014). Hot flashes are triggered by a slight rise in core body temperature, which leads to sympathetic nervous system activation. This occurs through peripheral vasodilation and increased sweat gland activity. The underlying mechanism may involve the hypothalamus response to reduced estrogen levels and the modulation of neurotransmitters such as serotonin and noradrenaline (Forma et al., 2024). Genitourinary syndrome of menopause (GSM) is a set of genital and urinary signs and symptoms associated with decreased estrogen. They represent chronic and progressive conditions that cause symptoms such as vaginal dryness and irritation, urinary problems, and sexual discomfort (Angelou et al., 2020). The pathophysiology of GSM is mainly due to the deficiency of a hormone known as estrogen, which leads to atrophy of vulvovaginal and urinary tissues. Estrogen plays a critical role in maintaining the normal physiology of the urogenital tissues, including epithelial thickness, collagen content, and vascularization. When estrogen levels decrease, these tissues lose their elasticity and lubrication, leading to symptoms like vaginal dryness, thinning of the epithelium, increased pH, and a shift in vaginal flora, which predisposes to infections and irritation. Additionally, estrogen receptors in the bladder and urethra are affected, contributing to urinary symptoms such as incontinence (Gandhi et al., 2016).

Osteopenia, a subclinical condition resulting in decreased bone density that often precedes osteoporosis, affects 50% of women over age 50 in the United States and is associated with a 1.8-fold increased risk of fracture compared with women with normal bone density. Available treatment options, including hormone therapy, bisphosphonates, and selective estrogen receptor modulators, can help prevent further bone loss, although concerns over rare but significant side effects often discourage prolonged use (Wright, 2014). Phytoestrogens, plant-derived compounds, have been studied to treat vasomotor symptoms of menopause. Some studies suggest good tolerance and associated benefits for the perimenopausal, menopausal, and postmenopausal phase, particularly with soy extracts and genistein. Phytoestrogens may improve bone mineral density and CVD risk markers, and there is no evidence that phytoestrogens increase the risk of breast, endometrial, or colorectal cancer (Rowe and Baber, 2021).

Concerns about the potential negative effects of hormone replacement therapy used for the treatment of menopausal symptoms have shifted attention to supplements composed of phytoestrogens. The word “phytoestrogen” comes from the Greek term for plant (“phyto-”) and the term “estrogen,” which is a sex hormone that influences female fertility in vertebrates. Phytoestrogens are compounds found in plants that, due to their molecular structure and size, resemble estrogens, particularly estradiol (17-β-estradiol, E2), and that can act in an estrogenic agonist or antagonist manner (Farquhar, 2005).

Polyphenols are a diverse group of naturally occurring compounds known for their antioxidant and health-promoting properties. Key examples include resveratrol, found in grapes, red wine, and berries, which supports heart health and has anti-inflammatory effects, and quercetin, abundant in onions, apples, and berries, known for its anti-inflammatory and antihistamine actions. Curcumin, the active component in turmeric, and epigallocatechin gallate (EGCG) in green tea, are both valued for their antioxidant and anti-inflammatory benefits, with EGCG linked to metabolic and cardiovascular health. Other important polyphenols are ellagic acid from berries, with potential anti-cancer effects, and catechins in green tea, cocoa, and apples, known for cardiovascular support. Anthocyanins, responsible for the red, blue, and purple colors in fruits like blueberries and grapes, offer cardiovascular and anti-inflammatory benefits, while flavonoids, widely found in citrus fruits, green tea, and berries, provide broad antioxidant support. Hesperidin, a bioflavonoid in citrus fruits, supports blood vessel health, and gallic acid, found in grapes, green tea, and berries, is noted for its antimicrobial and antioxidant effects. Together, these polyphenols contribute to improved overall health and disease prevention.

Polyphenols are the largest group of non-energetic substances present in plant-based foods with a high antioxidant capacity, which helps combat free radicals that are involved in the appearance of various diseases. A study highlights the beneficial effects of grape polyphenols in reducing cardiovascular risk factors in a menopause model. Ovariectomized guinea pigs, used to mimic postmenopausal conditions, exhibited elevated plasma triglycerides and cholesterol levels, as well as increased cholesterol accumulation in the aorta, which are factors associated with cardiovascular disease (CVD).

The administration of grape polyphenols significantly decreased plasma triglycerides and reduced cholesterol deposition in the aorta, suggesting a protective cardiovascular effect. Research demonstrates that grape polyphenols may help mitigate the heightened risk of CVD in postmenopausal states by improving lipid profiles and reducing cholesterol buildup in arterial walls (Zern et al., 2003).

The study on grape polyphenols cardioprotective effects in pre- and postmenopausal women found several beneficial outcomes. When participants consumed lyophilized grape powder (LGP), it led to significant reductions in plasma triglycerides, LDL cholesterol, and apolipoproteins B and E. Additionally, the study observed a decrease in cholesterol ester transfer protein activity and reduced whole-body oxidative stress, as indicated by lower urinary F (2)-isoprostane levels. The intake of LGP also lowered inflammatory markers such as plasma tumor necrosis factor-alpha (TNF-α), which plays a critical role in inflammation. Despite these positive effects, LGP did not significantly alter LDL oxidation. Overall, the findings suggest that grape polyphenols can improve key cardiovascular risk factors, likely through the modulation of lipids, oxidative stress, and inflammation, making them a potential therapeutic approach for reducing cardiovascular disease risk in pre- and postmenopausal women (Sandoval-Ramírez et al., 2017).

Flavonoids are a prominent group of plant compounds known for their antioxidant, anti-inflammatory, and immune-supporting properties, widely studied for their health benefits. Quercetin, found in apples, onions, and berries, is recognized for its strong anti-inflammatory and antihistamine effects. Catechins, present in green tea and cocoa, contribute to cardiovascular and metabolic health, while epicatechin, another type of catechin, supports heart health and may aid in lowering blood pressure. Hesperidin, a flavonoid found in citrus fruits like oranges and lemons, is beneficial for blood vessel health and can reduce inflammation. Kaempferol, found in broccoli, kale, and tea, has anti-cancer potential, as well as antioxidant and anti-inflammatory effects. Anthocyanins, the pigments giving red, blue, and purple color to fruits like blueberries, blackberries, and grapes, are linked to improved cardiovascular health and cognitive function. Lastly, myricetin in berries, tea, and red wine shows antioxidant, anti-diabetic, and anti-cancer properties. Collectively, flavonoids contribute to disease prevention and overall health by neutralizing free radicals, supporting cardiovascular health, and enhancing immune responses.

Isoflavones are a subclass of flavonoids primarily found in soybeans, legumes, and other plants, valued for their phytoestrogenic properties, which can mimic or modulate estrogen activity in the body. Key isoflavones include genistein and daidzein, predominantly found in soy, which have been widely studied for their potential to alleviate menopausal symptoms, such as hot flashes and bone loss, by binding to estrogen receptors. Biochanin A, present in red clover and chickpeas, offers antioxidant and anti-inflammatory effects, supporting cardiovascular and bone health. Glycitein, another soy isoflavone, contributes additional antioxidant protection and has been associated with improved lipid profiles. Isoflavones are particularly noted for their potential to support hormonal balance, offering a natural alternative to hormone therapy by providing mild estrogenic effects without the associated risks. As a group, isoflavones play a significant role in promoting bone health, cardiovascular protection, and possibly reducing risks of hormone-related cancers.

In ruminants, soy isoflavones (daidzein, genistein, glycitein) and red clover isoflavones (formononetin, biochanin A, daidzein, genistein) are metabolized to equol, which is a metabolically inactive p-ethylphenol (Woclawek-Potocka et al, 2013). The main dietary sources of isoflavones in human nutrition are soybeans and their products, which are rich in daidzein and genistein. When consumed, these isoflavones can exert estrogenic and/or antiestrogenic effects. Aglycones known as free flavonoids are absorbed in the proximal part of the small intestine by passive diffusion and reach maximum blood concentration one hour after infusion into the duodenum. Dietary supplements of phytoestrogens made from red clover extracts, which are becoming increasingly popular as an alternative therapy for the treatment of menopausal symptoms, indirectly provide a source of daidzein, since the methoxylated isoflavone formononetin from red clover is efficiently transformed to daidzein in the human gastrointestinal tract (Setchell, 2001).

Isoflavones are considered to have chemoprotective properties and can be used as an alternative therapy for various hormonal disorders, including several types of cancer, cardiovascular diseases, osteoporosis, and menopause-related symptoms. Conversely, they may also act as endocrine disruptors, which could have adverse effects on the health of certain populations or the environment. In general, the intake of dietary supplements containing isoflavones leads to a modest reduction in the frequency of hot flashes (10-20%). Stronger effects of isoflavones were observed in women with a higher frequency of hot flashes (Howes, 2006).

Resveratrol is a naturally occurring chemical or polyphenol found in red grapes and grape products, blueberries, raspberries, and blackberries, and to a lesser extent in peanuts. Resveratrol supplementation has been shown to improve menopause-related symptoms such as cognitive function, bone mineral density, and pain perception. It also improves cerebrovascular function in postmenopausal women. Significant effects on depression and insomnia were found when resveratrol was used in combination with other ingredients. Similarly, marginal improvements in mood states and a trend toward better overall well-being and quality of life were observed. Overall, resveratrol may offer a promising treatment for menopause-related complaints (Cooney et al., 2022).

A randomized, placebo-controlled study investigated the effects of equol and resveratrol supplementation on health-related quality of life in menopausal women. The study found that a daily dose of 200 mg of fermented soy containing 10 mg of equol and 25 mg of resveratrol was effective for 12 weeks in improving menopausal symptoms. The study found that supplementation significantly improved menopausal symptoms, particularly vasomotor symptoms (such as hot flashes) and psychological well-being. Improvements in physical functioning, energy levels, and overall quality of life were also observed. These results suggest that equol and resveratrol may be beneficial in managing menopausal symptoms and improving quality of life during menopause (Davinelli et al., 2017).

Quercetin is a plant pigment or flavonoid, found in many foods and plants, such as onions, green tea, apples, red wine, and berries. It is the most common flavonoid in the human diet with antioxidant effects. This flavonoid increases oxidative stress against free radicals in both menopausal rats in vivo and ovarian granulosa cell cultures in vitro. It helps preserve ovarian function and reduces oxidative damage associated with menopause, probably through interactions with estrogen receptors that maintain antioxidant defenses in reproductive tissues. Efficacy is observed at varying doses, with no single optimal dose being specified (Kiyama, 2023).

Myricetin is a flavonoid with antioxidant properties that has shown several potential benefits in managing menopausal symptoms, mainly through its antioxidant, anti-inflammatory, and bone-protective properties. It improves bone health by inhibiting bone resorption and enhancing osteogenic differentiation, crucial for preventing osteoporosis, a common concern in menopausal women. Furthermore, myricetins antioxidant capabilities help mitigate oxidative stress, which typically increases during menopause and contributes to various symptoms and complications. These properties suggest that myricetin may be beneficial in reducing the risk of cardiovascular disease and other oxidative stress-related conditions during menopause (Imran et al., 2021).

Cannabis sativa L. is an annual, usually dioecious, herbaceous plant in the family Cannabaceae, which grows freely in tropical and subtropical regions and has an extensive history of medical and therapeutic use. The growing anecdotal use of cannabis for managing menopause symptoms warrants greater attention (Babyn et al. 2023). Cannabis is marketed as a natural, organic remedy for various women health issues during menopause, including sleep disturbances, mood swings, and anxiety.

The endocannabinoid system (ECS) exists within various organ systems and is integral to the regulation of smooth muscle, immune responses, neuroendocrine function, and tissue metabolism. It includes cannabinoid receptors CB1 and CB2, the endocannabinoid agonists anandamide and 2-arachidonoylglycerol (2-AG), and the enzymes involved in their synthesis and metabolism (Lu & Mackie, 2021). As a central and peripheral neuromodulatory system, the ECS is characterized by two primary cannabinoid receptors: CB1 and CB2.

The CB1 receptor is predominantly expressed in the brain and peripheral tissues such as adipocytes, immune cells, skeletal muscle, the exocrine pancreas, liver, gastrointestinal tract, and reproductive organs. Conversely, CB2 receptors are primarily located in the spleen, thymus, pancreas, and peripheral immune cells, including mast cells and leukocytes, with a lower density of CB2 receptors in the central nervous system compared to CB1 receptors (Capodice & Kaplan, 2021).

Notably, CB1 receptors are most concentrated in the nervous system, where they are highly expressed in glutamatergic, cholinergic, glycinergic, and serotonergic neurons, especially at synaptic terminals. Cannabinoids stimulate the central nervous system, regulating the secretion of neurotransmitters such as dopamine and serotonin, and hormones such as oxytocin and estradiol. These hormones influence mood swings in menopause.

Although less prevalent, CB2 receptors can also be found in the central nervous system, particularly in microglia and other immune-origin cells. This broad distribution of cannabinoid receptors suggests that the endocannabinoid system is extremely intricate and multifunctional, interacting with multiple signaling pathways (including those of dopamine and opioids) and influencing a wide range of endogenous processes (Filipiuc, 2021).

Endocannabinoids exert a wide range of effects on various tissues, including those of the reproductive system. Growing evidence highlights the crucial role of endocannabinoids in reproductive processes, including folliculogenesis, spermatogenesis, fertilization, oviductal transport, implantation, and embryo development. The observed positive correlations between plasma anandamide (AEA) and serum follicle-stimulating hormone (FSH), luteinizing hormone (LH), and estradiol (E2) levels in premenopausal women suggest that these hormones might play a role in regulating AEA, or conversely, AEA could influence the levels of these hormones (El-Talatini et al., 2010).

A study aiming to investigate whether the absence of estrogen in ovariectomized rats, along with sex steroid replacement, would lead to changes in the expression of elements of the endocannabinoid system in the uterus found that the expression of ECS components, as well as anandamide (AEA) and prostaglandin E2 (PGE2) levels in the rat uterus, are modulated by estradiol benzoate (EB). Thus, estradiol may directly regulate the ECS in female reproductive tissues (Maia et al., 2017). These findings underscore the complex interplay between endocannabinoids and sex hormones in reproductive biology, suggesting potential therapeutic avenues for addressing reproductive health issues.

Endocannabinoids interact with the hypothalamic-pituitary-gonadal (HPG) axis by inhibiting the release of gonadotropin-releasing hormone (GnRH) and luteinizing hormone (LH), resulting in lower levels of gonadal hormones like testosterone, estradiol, and progesterone. In females, endocannabinoids reduce LH release, while estrogens influence endocannabinoid signaling by modulating cannabinoid receptor expression and altering the activity of enzymes such as fatty acid amide hydrolase (FAAH), which is involved in breaking down endocannabinoids. Similarly, progesterone regulates endocannabinoid metabolism, particularly affecting FAAH activity in reproductive tissues. These interactions form a feedback loop where endocannabinoid activity reduces gonadal hormone production, while gonadal hormones modulate the endocannabinoid system, impacting reproductive functions and behaviors. These insights have important implications for understanding and potentially treating hormone-related conditions (Gorzalka & Dang, 2012).

The endocannabinoid system (ECS) influences the estrogen system by regulating estrogen-related processes in both the central nervous system (CNS) and peripheral tissues. The ECS helps maintain homeostasis in functions such as synaptic plasticity, reproduction, and neuroendocrine regulation. Concerning estrogen, cannabinoids interact with estrogen receptors to modulate processes like mood regulation, reproductive health, and neuroprotection. In cases of estrogen deficiency, such as during menopause, cannabinoids could be administered to alleviate symptoms.

Phytocannabinoids, like cannabidiol (CBD) may help restore balance in estrogen-related functions. These cannabinoids can be administered in various forms, such as oral supplements or oils, and may aid in addressing symptoms of estrogen deficiency by interacting with the ECS to regulate hormone levels and support physiological processes affected by low estrogen (Santoro et al., 2021).

For instance, one study investigated how estrogen might interact with the endocannabinoid system, particularly through the enzyme FAAH, to influence emotional behavior in ovariectomized rats. Researchers tested this by giving ovariectomized rats either estrogen or a control substance, and then an hour later, either a blocker of cannabinoid receptors or a control. The rats were then observed in different behavioral tests. Rats that received estrogen spent more time in areas of the tests that indicated reduced anxiety, but the cannabinoid blocker reversed this effect. This suggests that the mood-lifting effects of estrogen might be linked to the endocannabinoid system. Another test showed that blocking FAAH led to less anxiety and depression-like behavior in the rats. Overall, these findings imply that estrogens effects on mood may be tied to the ECS and that targeting FAAH could be a way to treat anxiety and depression in women (Hill et al., 2007).

There was a study aiming to assess whether hemp seed can prevent hypercholesterolemia induced by ovarian hormone deficiency in ovariectomized (OVX) rats, a model for menopause. The results showed that rats in the hemp seed treated (HST) group at 1% and HST2% groups lost weight, while those in the sham and OVX groups gained weight; the HST10% group maintained a stable weight. Additionally, the study aimed to evaluate the effects of hemp seed on complications related to estrogen deficiency, including depression, anxiety, and potential cognitive impairments. The forced swimming test assesses depressive-like behavior in animals, with decreased mobility or shorter swim times indicating higher despair.

Fecal boli count measures anxiety levels, where fewer boli suggest reduced stress. A reduction in exploratory diving percent signifies increased exploratory behavior, often interpreted as a sign of decreased anxiety or fearfulness. Plasma estradiol levels in the HST groups were restored to levels like those of the sham group, and these rats exhibited longer swim times compared to the OVX group, with the HST 10% group achieving the longest swim times. Additionally, hemp seed treatment led to a significant decrease in fecal boli numbers and a reduction in exploratory diving percent, with the HST10% group demonstrating the most pronounced effects (Saberivand et al., 2010).

On the other hand, a study aimed to investigate how AEA affects the heme oxygenase (HO) and nitric oxide synthase (NOS) systems in rats with depleted estrogen levels. Female Wistar rats that were either sham-operated (SO) or underwent OVX to induce estrogen deficiency were used in the study. Some OVX rats were given 0.1 mg/kg of estrogen (E2) orally for two weeks, while the effects of AEA were analyzed after two weeks of treatment. By the end of the experiment, various parameters, including cardiac activity, HO and NOS enzyme expression, cannabinoid 1 receptor content, and concentrations of transient potential vanilloid 1 (TRPV1) and calcitonin gene-related peptide (CGRP), were measured.

Results showed that the withdrawal of estrogen significantly decreased the NOS and HO systems and affected the TRPV1/CGRP pathway. Treatment with either AEA or E2 for two weeks reversed these adverse changes. These findings suggest that the endocannabinoid system is involved in regulating cardiovascular signaling pathways and may serve as a potential therapeutic target for cardiac issues related to menopause. Furthermore, AEA produced effects similar to the estrogen replacement therapy, which suggests AEA estrogen-like role in this model (Szabó et al., 2020). Preclinical and early clinical work suggests that cannabis-derived compounds (phytocannabinoids) may have an impact on bone turnover. Cannabidiol (CBD) is a non-intoxicating cannabinoid that may be involved in maintaining bone integrity by reducing osteoclastic bone resorption (Whyte L, 2009).

A study focused on cannabidiol (CBD) treatment in ovariectomized (OVX) mice revealed several significant benefits for glucose, energy, and bone metabolism. CBD enhanced oral glucose tolerance and increased energy expenditure, particularly through improved oxygen utilization. The treatment had a positive effect on bone density and microstructure, reversing the OVX-related loss of bone mineral density (BMD), increasing trabecular bone volume, and decreasing bone resorption.

Additionally, CBD treatment resulted in lower pro-inflammatory markers in both gut and bone tissue, indicating reduced inflammation. The study also noted a rise in beneficial gut bacteria, especially Lactobacillus species, which are associated with improved bone health and metabolic function. These findings suggest that CBD could be a potential therapeutic option for counteracting some of the metabolic and bone-related issues linked to estrogen deficiency in postmenopausal women (Sui et al., 2022).

A case series involving two postmenopausal women with osteopenia evaluated the effects of oral cannabidiol (CBD) treatment. Participants received daily doses of either 100 mg or 300 mg of CBD over 12 weeks. The treatment led to a reduction in bone turnover markers, indicating that CBD may help slow bone loss in postmenopausal women, offering a potential therapeutic option for managing osteopenia (Kulpa et al., 2023).

Regarding the effects of CBD on mood disturbances, researchers explored the acute systemic administration of CBD in Swiss mice subjected to the forced swim test (FST), a widely recognized test for predicting antidepressant effects. The results demonstrated that CBD induces dose-dependent antidepressant effects, with the effective dose being 30 mg/kg. These effects were blocked when the 5-HT1A receptor antagonist WAY100635 was administered beforehand, indicating that CBDs antidepressant action is mediated through 5-HT1A receptor activation.

Further research showed that a single systemic administration of CBD produced similar antidepressant effects in Swiss mice, both in the FST and the tail suspension test (TST), although at significantly higher doses (200 mg/kg). Importantly, CBD also proved effective after repeated administration, as observed in albino Swiss mice, where daily injections of CBD at doses of 3 and 30 mg/kg for 15 days exhibited antidepressant-like effects in the TST (Zanelati et al., 2010).

Cannabigerol (CBG) has shown promising potential as both an antidepressant and an antioxidant. In terms of its effects on depression, a survey by Russo et al. (2022) found that users predominantly consuming CBG for medical purposes reported significant improvement in depression symptoms, with 80% of participants claiming that CBG was more effective than conventional medications. Additionally, CBG acts as a potent antagonist of the 5-HT1A receptor, a key target in depression treatment, which may suggest its involvement in modulating serotonergic signaling. As an antioxidant, CBG has demonstrated the ability to counteract oxidative stress and regulate various signaling pathways. Studies have shown that CBG reduces oxidative stress by inhibiting the production of reactive oxygen species (ROS) and regulating pro-oxidative markers such as iNOS and nitrotyrosine.

The analgesic and anti-inflammatory effects of cannabinol (CBN) have been extensively studied. CBN has been shown to exhibit analgesic activity in various in vivo tests. For example, in the Randall-Selitto paw pressure test and the acetic acid-induced writhing test, CBN demonstrated dose-dependent analgesic effects, similar to aspirin. It was effective in reducing the frequency of writhing and increasing the pain threshold in non-injected paws. Moreover, CBN displayed anti-inflammatory properties in vitro by significantly inhibiting pro-inflammatory cytokines like IL-6, IL-10, and MCP-1 in lipopolysaccharide (LPS)-stimulated U937 cells, although it was less responsive to IL-1B, IL-8, and TNF-α. These findings suggest that CBN may offer potential benefits as an analgesic and anti-inflammatory agent, particularly through its actions on CB2 receptors and its ability to modulate peripheral inflammation (Khouchlaa et al., 2024).

It has been described that the use of cannabinoids positively influences the estrogenic response since it has been observed that cannabinoids interact with cellular estrogen receptors and can induce their hormonal effect on women. A study evaluating the pattern of use and perceptions about cannabis for menopause in women aged 35 and over, showed that among the cannabis users, more than 75% used it for medical purposes. The primary reasons included sleep (65%), anxiety (45%), and muscle/joint achiness (33%). Additionally, 74% of these users reported that cannabis was effective in alleviating their symptoms. Therefore, most current cannabis users found it beneficial to manage menopause symptoms (Babyn et al., 2023).

Another study explored the use of medical cannabis (MC) for alleviating menopause-related symptoms in perimenopausal and postmenopausal individuals. Participants, including 131 perimenopausal and 127 postmenopausal individuals, reported on their menopause symptoms and cannabis usage, specifying modes of consumption, types of use, and targeted symptoms. A significant majority of participants were current cannabis users (86.1%) and used MC to address menopause symptoms (78.7%), with smoking (84.3%) and edibles (78.3%) being the most common modes of use. Sleep disturbances (67.4%) and mood/anxiety issues (46.1%) were the top symptoms targeted by MC use. Perimenopausal participants reported more severe menopause-related symptoms on vasomotor and psychosocial scales compared to postmenopausal participants, with a higher burden of anxiety, hot flashes, and a greater incidence of depression and anxiety diagnoses. They also reported increased use of MC for mood and anxiety symptoms. The findings indicate a widespread use of MC as a supplementary treatment for menopause symptoms, especially sleep and mood issues, and suggest that cannabinoid-based therapies could be a promising area for clinical trials. Future research should focus on the impact of different MC characteristics, such as cannabinoid profiles, on treatment effectiveness (Dahlgren et al., 2022).

Among the benefits of cannabis use in vaginal dryness that occurs during menopause, there is evidence of increased female lubrication, increased libido and greater feeling of fullness. All of these processes are mediated by cannabinoid cell receptors of the CB1 type, which demonstrates the breadth and scope of the endocannabinoid system in the various aspects of women sexuality (Androvicova J., et al., 2017).

A systematic review on cannabidiol (CBD) dosing in clinical populations suggests that the appropriate CBD dosage can vary depending on the condition being treated. For most clinical applications, doses range between 20 mg to 1500 mg per day, administered orally. However, it was emphasized that the dosing varies widely across different conditions, with lower doses (around 20-400 mg/day) often used for conditions like anxiety, while higher doses (up to 1500 mg/day) are more common in conditions such as epilepsy (Millar et al., 2019).

Overall, positive results of cannabis in managing mood and sleep disturbances, as well as vasomotor symptoms, have been observed in rat models of menopause. Moreover, studies in menopausal women using cannabis have also shown good results in managing the associated symptoms of mood and sleep disturbances.

### Pharmaceutical Compositions

Aspects of the disclosure provide a method to treat symptoms associated with menopause and maintain optimal hormonal health in women using a nanoplatform composition using nanoemulsion of phytocannabinoids: (CBD, CBG, CBN), flavonoids and polyphenols specifically aimed at patients who are going through menopause as a result of hormonal cessation associated with ovarian dysfunction that comes with age as well as patients who are suffering from this pathology as a result of surgical interventions such as the removal of ovaries and consequently hormonal cessation.

The disclosure, as described herein as embodied in the examples provided, is not limited to the details shown because various modifications and changes can be made without departing from the scope of the disclosure and the equivalent of the claims. The following description of specific embodiments will help clarify the construction, operation, and additional benefits of the disclosure. Aspects of the disclosure refer to a composition for treatment of menopause syndrome, comprising: a) an active principle comprising at least one cannabinoid; and b) a stabilizing solubilizing agent.

In some embodiments, the active principle comprises at least one cannabinoid among Δ9-Tetrahydrocannabinol (THC), Cannabidiol (CBD), Cannabigerol (CBG), Cannabinol (CBN), Tetrahydrocannabivarin (THCV), Cannabichromene (CBC), Delta-8-Tetrahydrocannabinol (Δ8-THC), Cannabidivarin (CBDV), Tetrahydrocannabinolic Acid (THCA), Cannabidiolic Acid (CBDA), Cannabigerolic Acid (CBGA), Cannabichromenic Acid (CBCA).

These cannabinoids may be used individually or in combination to achieve a synergistic effect, targeting specific therapeutic outcomes. The selection of one or more cannabinoids can be tailored to address specific physiological conditions, such as managing pain, inflammation, mood disorders, or neuroprotection. The cannabinoid composition may also be optimized in certain embodiments to leverage the entourage effect, wherein the combined action of multiple cannabinoids and terpenes enhances overall efficacy, providing a more comprehensive therapeutic benefit for conditions associated with the menopausal transition or other targeted medical conditions.

In various embodiments, the composition may vary in the concentration of cannabinoids. Additionally, the ratio of different cannabinoids may be varied to optimize the therapeutic profile while maintaining efficacy. In further embodiments, the composition may combine two or more cannabinoids in specific ratios to produce a synergistic effect, where the combined therapeutic action is greater than the effect of individual cannabinoids alone.

In various embodiments, the composition may vary in the concentration of cannabinoids to tailor therapeutic potency and patient response, allowing for precise adjustment based on the condition being treated, patient tolerance, and desired therapeutic outcome. For example, higher concentrations of cannabidiol (CBD) may be employed for its anti-inflammatory and anxiolytic properties, whereas compositions with increased levels of tetrahydrocannabinol (THC) might be suited for enhanced analgesic effects in patients managing chronic pain.

Additionally, the ratio of different cannabinoids within the composition can be adjusted to optimize the therapeutic profile, balancing efficacy with minimized side effects. This approach allows the composition to be customized, such as formulating a high-CBD, low-THC ratio to reduce psychoactive effects while still delivering anti-inflammatory benefits or using a balanced ratio of CBD and cannabigerol (CBG) to potentially enhance neuroprotective effects. The composition includes cannabinoids at a concentration ranging from 4% to 20%. In a preferred embodiment, composition includes cannabinoids at a concentration ranging from 5% to 15%. In a further preferred embodiment, composition includes cannabinoids at a concentration ranging from 5% to 12%.

In further embodiments, the composition may combine two or more cannabinoids in specific ratios that yield a synergistic effect, where the therapeutic action of the combination exceeds the benefits of each cannabinoid individually. This synergistic effect, often referred to as the “entourage effect,” enables the composition to address complex conditions more effectively by modulating various physiological pathways simultaneously. For instance, combining CBD and THC in a particular ratio could potentiate pain relief while reducing inflammation, and incorporating minor cannabinoids such as tetrahydrocannabivarin (THCV) or cannabichromene (CBC) may further refine the composition efficacy in areas like appetite control, mood stabilization, and bone health.

In further embodiments, the composition may combine two or more cannabinoids in specific ratios, such as a high CBD-to-THC ratio for anti-inflammatory and anxiolytic effects with minimal psychoactivity, or a balanced CBD-CBG formulation to enhance neuroprotective benefits. These tailored ratios aim to produce a synergistic effect, where the combined therapeutic action of cannabinoids is greater than the effect of individual cannabinoids alone. Additionally, in certain embodiments, minor cannabinoids, such as THCV or CBC, may be included in precise quantities alongside primary cannabinoids to modulate effects like appetite suppression, cognitive function, or bone health, thus providing a more comprehensive therapeutic benefit for conditions associated with menopause, chronic pain, or other targeted conditions.

Compositions of the disclosure may be presented in any pharmaceutical dosage forms such as solid, semisolid, liquid and gas forms. In one embodiment the pharmaceutical dosage form may be tablets, capsules, powder, dusting powder, or other related solid forms. In another embodiment, the pharmaceutical dosage form may be cream paste, gel, suppositories or other related semi solid forms. In another embodiment, the pharmaceutical dosage form may be syrup, solution, emulsion, suspension, or other related liquid forms. In another embodiment, the pharmaceutical dosage form may be inhaler, aerosols or other related gas forms. In a further preferred embodiment, the pharmaceutical dosage form may be capsules or suppositories.

Compositions of the disclosure may be presented in various pharmaceutical dosage forms, accommodating diverse routes of administration to optimize patient compliance and therapeutic effectiveness. These forms include solid, semisolid, liquid, and gaseous formulations, each designed to meet specific therapeutic needs and target different administration pathways. In one embodiment, the pharmaceutical dosage form may be a solid form, such as tablets, capsules, powders, or dusting powders, which are convenient for oral administration and suitable for controlled-release formulations. These solid forms allow precise dosing and are often preferred for their stability and ease of transport and storage.

In another embodiment, the composition may be formulated as a semisolid, such as creams, pastes, gels, or suppositories, which provide localized or systemic absorption. Semisolid forms are particularly advantageous for topical or intravaginal administration, allowing the active ingredients to act directly at the site of application for targeted relief, such as alleviating genitourinary symptoms of menopause.

In another embodiment, the pharmaceutical dosage form may be a liquid, including syrups, solutions, emulsions, or suspensions, which are suitable for oral, sublingual, or parenteral administration. Liquid formulations offer flexibility in dosing and rapid absorption, making them ideal for individuals requiring swift symptom relief or for patients who have difficulty swallowing solid forms.

In an additional embodiment, the composition may be administered in a gaseous form, such as through inhalers or aerosols, which are suitable for pulmonary delivery. This form allows for rapid absorption of cannabinoids via the respiratory tract, providing an efficient delivery method for systemic effects with fast onset, useful in managing acute symptoms.

In a further preferred embodiment, the pharmaceutical dosage form may be either capsules or suppositories. Capsules provide a precise and controlled oral dosing option, while suppositories allow for rectal or vaginal administration, offering a localized delivery route with minimal systemic side effects. These preferred forms enhance bioavailability and provide versatile options for both systemic and localized therapeutic effects tailored to the specific needs of menopausal and postmenopausal women.

In some embodiments, the included cannabinoids are CBD (cannabidiol), CBG (cannabigerol), CBN (cannabinol) or combinations thereof. In some embodiments, the composition includes cannabinoids at a concentration ranging from CBD 8%-11%, CBG 0.4%-0.6%, CBN 0.15%-0.30% in the capsules and CBD 5%-10% in the ovules.

In some embodiments, the included cannabinoids are cannabidiol (CBD), cannabigerol (CBG), cannabinol (CBN), or combinations thereof. These cannabinoids may be selected for their individual or synergistic therapeutic properties, with CBD known for its anti-inflammatory, anxiolytic, and analgesic effects, CBG for its neuroprotective and anti-bacterial properties, and CBN for its sedative and sleep-promoting benefits.

In some embodiments, the composition is formulated with specific concentrations of cannabinoids, carefully balanced to optimize therapeutic outcomes. For instance, the composition may include cannabinoids in the following concentration ranges: CBD at 8%-11%, CBG at 0.4%-0.6%, and CBN at 0.15%-0.30% in oral capsules. These concentrations are designed to deliver effective relief for menopausal symptoms such as pain, mood disturbances, and sleep disorders while minimizing psychoactive effects.

Additionally, in some embodiments, the composition may include cannabinoids in vaginal ovules, with CBD concentrations ranging from 5%-10%. These ovules are formulated to provide localized relief from genitourinary syndrome of menopause (GSM), including symptoms like vaginal dryness, irritation, and discomfort. The use of ovules allows for targeted delivery of CBD, maximizing local absorption while reducing systemic exposure.

In further embodiments, the ratio of cannabinoids can be adjusted within these concentration ranges to tailor the composition to specific patient needs or therapeutic goals, providing flexibility in managing a wide spectrum of menopausal and postmenopausal symptoms. These variations in cannabinoid content across different delivery systems, such as capsules and ovules, offer a comprehensive and versatile approach to symptom relief, addressing both systemic and localized needs. In another embodiment, the composition may be combined with herbal extracts, incorporating other plant-derived compounds such as polyphenols, flavonoids, isoflavones and combinations thereof, which may enhance the therapeutic potential of the cannabinoids through the entourage effect.

In one embodiment, the polyphenols comprises resveratrol, quercetin, curcumin, epigallocatechin gallate (EGCG), ellagic acid, catechins, anthocyanins, hesperidin, gallic acid and combinations thereof. In a preferred embodiment, the polyphenols comprises resveratrol. In further embodiments, the flavonoids comprises quercetin, catechins, epicatechin, hesperidin, kaempferol, anthocyanins, myricetin and combinations thereof. In a preferred embodiment, the flavonoids comprises Quercetin, Myricetin and combinations thereof. In further embodiments, the Isoflavones comprises soy isoflavones, particularly, genistein, daidzein, biochanin A, glycitein and combinations thereof.

In one embodiment, the pharmaceutical composition may further include various excipients or carriers to enhance stability, bioavailability, and patient acceptability. In further embodiments, these excipients could contain solubilizing, stabilizing solubilizing agents, emulsifying agents, antioxidants, and preservants. In further embodiments, these excipients could contain solubilizing agents such as polyethylene glycol (PEG) and propylene glycol; stabilizing agents like magnesium stearate and microcrystalline cellulose; emulsifying agents such as lecithin and polysorbates (e.g., polysorbate 80); antioxidants including ascorbic acid and tocopherols; and preservatives like benzyl alcohol and potassium sorbate.

In one embodiment, the pharmaceutical composition may further include various excipients or carriers to enhance stability, bioavailability, and patient acceptability, supporting both the efficacy and shelf life of the formulation. These excipients are chosen based on their ability to facilitate the absorption and distribution of the active cannabinoids while maintaining the overall integrity of the composition.

In further embodiments, these excipients may include solubilizing agents, stabilizers, emulsifiers, antioxidants, and preservatives. Solubilizing agents, such as polyethylene glycol (PEG) and propylene glycol, improve the solubility and absorption of cannabinoids, particularly for oral and intravaginal formulations, aiding in consistent therapeutic delivery. Stabilizing agents, such as magnesium stearate and microcrystalline cellulose, are included to ensure formulation consistency and prevent ingredient separation or degradation, especially in solid dosage forms like tablets or capsules.

Emulsifying agents, such as lecithin and polysorbates (e.g., polysorbate 80), are essential for creating homogeneous mixtures, particularly in liquid or semi-solid formulations, ensuring even distribution of cannabinoids and enhancing absorption when used in creams or gels. Antioxidants, including ascorbic acid and tocopherols, protect the composition from oxidative degradation, preserving the potency of cannabinoids over time.

Preservatives, such as benzyl alcohol and potassium sorbate, extend the shelf life of the formulation by preventing microbial growth, which is particularly important in semi-solid and liquid forms. In some embodiments, other excipients like buffering agents (e.g., citric acid) and chelating agents (e.g., EDTA) may be included to further stabilize the pH and prevent metal-induced oxidation, contributing to enhanced stability and prolonged efficacy.

Together, these carefully selected excipients ensure that the pharmaceutical composition remains safe, effective, and palatable, supporting optimal therapeutic outcomes and maximizing patient compliance across different administration routes, whether oral, topical, or intravaginal.

In this embodiment, the stabilizing agents, solubilizing agents, emulsifying agents, lipid agents, and preservatives in the capsule formulation are present at a concentration ranging from 9% to 15%. In another embodiment, the stabilizing and solubilizing agents, along with the oil base in the ovules, are present at a concentration ranging from 90% to 95%.

In one embodiment, the stabilizing and solubilizing agents for the capsule may include PEG-400, sorbitan, polysorbate, lecithin, sodium lauryl sulfate, poloxamers, or combinations thereof. In a preferred embodiment, the stabilizing and solubilizing agents comprise a mixture of PEG-400, sorbitan, and polysorbate. The concentration of these agents in the capsule ranges from 6.91% to 9.33%, with a preferred concentration of 6.91%.

Further embodiments may utilize an emulsifying system that includes agents such as PEG-400, sorbitan, polysorbate, lecithin, sodium lauryl sulfate, and poloxamers. In a specific formulation, the emulsifier system comprises sorbitan 80 at 1.4% to 2.8% and Tween 80 at the same proportion, with PEG-400 functioning as an auxiliary emulsifier at a concentration of 0.67% to 1.33%. In an additional embodiment, Span 80, Tween 80, and PEG-400 are incorporated as surfactants to enhance the emulsification and stability of the cannabinoid preparation during formulation.

In one embodiment, the antimicrobial preservative in the capsule may include propylparaben, methylparaben, sodium benzoate, benzoic acid, sorbic acid, potassium sorbate, or mixtures thereof. In a preferred embodiment, the antimicrobial preservative is a combination of propylparaben and methylparaben. The antimicrobial preservative in the capsule may be present at a concentration ranging from 0.1% to 0.2%, with a preferred concentration of 0.2%.

In further embodiments, the lipid agents may include, but are not limited to, medium-chain triglycerides (MCT oil), mineral oil, olive oil, cocoa butter, or combinations thereof. These lipid agents are selected to enhance the solubility and bioavailability of cannabinoids within the composition. In some embodiments, the medium-chain triglycerides (MCT oil) are present in a concentration ranging from 11% to 15%, a range shown to significantly benefit cannabinoid dissolution and absorption. The use of MCT oil, known for its efficient delivery of lipophilic compounds, provides a stable medium that supports the targeted release and absorption of active cannabinoids, ultimately improving the therapeutic efficacy of the formulation.

In further embodiments, the antioxidants may include, but are not limited to, Vitamin E, EDTA (ethylenediaminetetraacetic acid), Vitamin C, or combinations thereof. These antioxidants are selected to stabilize the formulation by protecting cannabinoids from oxidative degradation, thereby preserving potency and extending shelf life. In certain embodiments, Vitamin E is included at a concentration ranging from 0.07% to 0.10%. Vitamin E not only acts as a protective antioxidant to prevent cannabinoid breakdown but also enhances their absorption and bioavailability by supporting the integrity of lipid-based carriers within the formulation. The incorporation of antioxidants like Vitamin E and Vitamin C serves to maintain the therapeutic effectiveness of the cannabinoids over time, ensuring reliable efficacy.

In further embodiments, the composition may incorporate an aqueous phase vehicle, which is essential for balancing the formulation and enhancing the solubility of active ingredients. The aqueous phase vehicle, present at a concentration ranging from 16.00% to 24.00%, may include saline phosphate buffer, purified water, or similar hydrating agents. This component helps to maintain the stability of the formulation, improve the consistency of dosage delivery, and support the absorption of cannabinoids, particularly in formulations where a water-based carrier enhances bioavailability and patient comfort.

In one embodiment, the composition can be formulated for multiple administration forms, may be prepared in various pharmaceutical forms, and may be designed to have different controlled release options to accommodate different therapeutic needs. In one embodiment, the phytocannabinoid-based pharmaceutical composition is designed for multiple routes of administration and therapeutic applications which may include: oral, sublingual, topic, inhalation, parenteral, and intravaginal offering flexibility in treatment and ensuring precise control over dosage and bioavailability to meet diverse patient needs.

In one embodiment, the phytocannabinoid-based composition is formulated into oral dosage forms, such as tablets, capsules, oral drops, syrups, suspensions, powders for oral reconstitution, elixir, creams, emulsions, oral solutions, gummies, caramels, sprays, oral topics, mouth rinses. In one embodiment, sublingual presentations such as capsules and drops, tablets, or films are developed for rapid absorption through the mucous membranes, bypassing first-pass metabolism and providing quicker therapeutic effects.

In one embodiment, for intravaginal administration, the pharmaceutical forms may include ovules, creams, or suppositories specifically designed for rapid absorption through the mucous membranes. These forms allow the active ingredients to be absorbed directly into the local tissue, bypassing first-pass hepatic metabolism, and thus delivering therapeutic effects more quickly and efficiently. This route of administration is particularly advantageous for targeting genitourinary symptoms associated with menopause, such as dryness, irritation, and discomfort, providing both localized relief and systemic benefits. The formulation of these intravaginal pharmaceutical forms is optimized to enhance mucosal permeability, ensure even distribution of active ingredients, and maintain patient comfort, making them an ideal choice for addressing menopausal and postmenopausal symptoms.

In further embodiments, the composition is provided as injectable solutions or suspensions, designed for intravenous, intramuscular, or subcutaneous administration. These forms offer precise and immediate delivery in settings where rapid cannabinoid action is needed. In some embodiments, these pharmaceutical forms may include immediate-release, sustained-release, or controlled-release options, allowing for precise regulation of cannabinoid bioavailability over time. Immediate-release formulations are designed to deliver cannabinoids quickly, providing rapid onset of relief for acute symptoms. Sustained-release forms gradually release the active ingredients over an extended period, which can help maintain therapeutic levels and reduce the need for frequent dosing. Controlled-release options offer even more targeted delivery by maintaining a steady release rate, optimizing cannabinoid levels in the system to manage chronic symptoms effectively and enhance patient adherence. By incorporating these release mechanisms, the composition can be tailored to meet a range of therapeutic needs, from immediate relief to long-term management of menopausal and postmenopausal symptoms.

### Kit for the Treatment of Menopause Symptoms

The present disclosure introduces a comprehensive therapeutic kit designed for the treatment and management of menopausal symptoms in women, leveraging a cannabinoid-based formulation in conjunction with polyphenols, flavonoids, and isoflavones. This kit offers an integrated solution to address the range of physiological and psychological challenges women experience during the menopausal transition and post-menopausal phases. The kit targets common symptoms such as vasomotor instability (e.g., hot flashes), genitourinary syndrome of menopause (GSM), mood disturbances, sleep disorders, and bone health, while also providing potential cardiovascular benefits.

### Key Components of the Kit

The therapeutic kit comprises two main products: Oral Capsules: Designed for systemic relief from menopausal symptoms such as hot flashes, mood swings, sleep disturbances, and cardiovascular health. Intravaginal Ovules: Developed to target localized symptoms such as vaginal dryness, burning, discomfort, and genitourinary syndrome of menopause (GSM), delivering cannabinoids directly to affected areas. Together, these two formulations offer a holistic approach to menopause care, addressing both systemic and localized symptoms through distinct, but complementary, modes of administration.

Oral Capsules: The oral capsules in the kit provide a nanoplatform cannabinoid-based formulation designed for daily use. The capsules contain a precise combination of active cannabinoids, polyphenols, flavonoids, and isoflavones, each contributing to a multi-dimensional approach to symptom management.

Active Cannabinoids: Cannabidiol (CBD), Cannabigerol (CBG), and Cannabinol (CBN) are the primary active cannabinoids in the capsules. These cannabinoids work through the endocannabinoid system to regulate mood, pain, and inflammation, making them effective for treating symptoms such as hot flashes, mood swings, and sleep disturbances.

Flavonoids and Polyphenols: The addition of polyphenols (e.g., resveratrol) and flavonoids (e.g., quercetin, myricetin) provides additional benefits, including antioxidant, anti-inflammatory, and cardioprotective effects. Resveratrol is noted for its ability to improve endothelial function and reduce the risk of cardiovascular disease (CVD), which is a growing concern for postmenopausal women due to declining estrogen levels.

Isoflavones: Isoflavones, particularly from soy, mimic the mild estrogenic effects in the body, contributing to the reduction of vasomotor symptoms (such as hot flashes) and supporting bone health, thereby reducing the risk of osteoporosis.

Capsule Composition: Each oral capsule includes the following active ingredients: CBD: 100 mg; CBG: 5 mg; CBN: 2.5 mg; Resveratrol: 70 mg; Quercetin: 250 mg; Myricetin: 50 mg; Isoflavones: 60 mg.

The formulation is optimized using nanoemulsion technology to improve bioavailability and therapeutic efficacy, ensuring that the active compounds are absorbed effectively within the body. By using a nano-sized platform, the cannabinoids and other active ingredients can cross biological barriers more easily, resulting in faster onset of action and sustained therapeutic benefits.

### Dosage and Administration:

The recommended regimen is one capsule in the morning and one capsule in the evening, taken daily for a duration of 12 weeks. The nanoemulsion technology ensures that even with daily administration, the bioavailability remains high, reducing the frequency of dosing and enhancing patient compliance.

Intravaginal Ovules: The second component of the kit consists of intravaginal ovules, specifically formulated to treat genitourinary symptoms associated with menopause. These symptoms, often grouped under the term genitourinary syndrome of menopause (GSM), include vaginal dryness, burning, irritation, and discomfort during sexual intercourse. The ovules are designed to be administered directly to the vaginal area, providing localized relief and improving the quality of life for women experiencing GSM.

CBD: Cannabidiol (CBD) serves as the primary active ingredient in the ovules, offering anti-inflammatory, analgesic, and lubricating properties. CBDs interaction with local endocannabinoid receptors (CB1 and CB2) helps to reduce inflammation and pain, improve tissue elasticity, and promote lubrication, thereby alleviating vaginal dryness and irritation.

Lipid-Based Formulation: The ovules use medium-chain triglycerides (MCTs) and cocoa butter as lipid carriers, both of which enhance the solubility and absorption of cannabinoids. The lipid base not only provides a soothing and lubricating effect but also ensures that the CBD is delivered effectively to the vaginal tissues, offering relief from GSM symptoms.

Vitamin E: Included for its antioxidant properties, Vitamin E further enhances the formulation by supporting cell regeneration and tissue health, helping to maintain the integrity of the vaginal epithelium.

Ovule Composition: Each ovule contains: CBD: 100 mg; Medium-chain triglycerides (MCT): 32.5%; Cocoa butter: 62.4%; Vitamin E: 0.1%

Dosage and Administration: The intravaginal ovules are administered once daily for a period of 12 weeks, delivering localized relief to the affected area. This targeted delivery system ensures that the therapeutic benefits of CBD are concentrated in the vaginal tissues, reducing the risk of systemic side effects.

In some embodiments, the purpose of the kit is to provide systemic and localized relief of menopause symptoms, offering a natural approach to improving the quality of life during the menopausal period transition. In a preferred embodiment, the disclosure comprises a kit designed for the comprehensive treatment of menopause symptoms, consisting of two products: capsules and ovules:

Product 1—capsules: is a composition comprising cannabinoids, flavonoids, isoflavones and polyphenols, specifically: CBD: 100 mg; CBG: 5 mg; CBN: 2.5 mg; Resveratrol: 70 mg; Quercetin: 250 mg; Myricetin: 50 mg; Isoflavones: 60 mg.

In further embodiments, the composition will be administered orally in the form of 2 capsules, each containing a concentration of: CBD: 100 mg, CBG: 5 mg, CBN: 2.5 mg, Resveratrol: 70 mg, Quercetin: 250 mg, Myricetin: 50 mg, Isoflavones: 60 mg. This treatment will be taken daily, 1 capsule in the morning and 1 capsule at night for 12 weeks. The capsules are developed using a nanoemulsion technique to improve the bioavailability and efficacy of the active compounds.

In one non-limiting embodiment, this composition for menopause care contains the following active ingredients per dosage: CBD-100 mg; CBG-5 mg; CBN-2.5 mg. In one preferred embodiment, the composition comprises the following concentrations: CBD at 10.64%, CBG at 0.53%, and CBN at 0.27%. In a further embodiment, the flavonoids in the composition include isoflavones, quercetin, and myricetin, while the polyphenols include resveratrol.

In one embodiment, the composition includes resveratrol at a concentration ranging from 5.50% to 7.50% per capsule. In another embodiment, the composition includes quercetin at a concentration ranging from 20% to 27% per capsule. In a further embodiment, the composition includes myricetin at a concentration ranging from 4% to 6% per capsule.

In an additional embodiment, the composition includes isoflavonoids at a concentration ranging from 5% to 7% per capsule. In some embodiments, the composition includes cannabinoids, flavonoids, and polyphenols as active principles. In a further embodiment, the composition comprises cannabinoids ranging from 8% to 13% and flavonoids ranging from 35% to 46% per capsule. In a preferred embodiment, the composition comprises cannabinoids such as CBD, CBG, and CBN, and flavonoids and polyphenols such as isoflavones, myricetin, quercetin, and resveratrol. In a further preferred embodiment, the composition comprises CBD 10.64%, CBG 0.53%, CBN 0.27%, isoflavones 6.38%, myricetin 5.32%, quercetin 26.6%, and resveratrol 7.45%.

In another embodiment, the composition additionally contains stabilizing agents and antioxidant agents. In a further embodiment, the stabilizing agents range from 6.91% to 9.33%, and the antioxidants from 0.07% to 0.10%. In a preferred embodiment, the composition comprises stabilizing agents such as PEG-400 and Tween 80, and antioxidants such as vitamin E. In a further preferred embodiment, the composition comprises PEG-400 at 2.13%, Tween at 2.66%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for oral administration and presented in capsules.

Product 2—ovules: is formulated to address vaginal dryness and contains: CBD: 100 mg.

In some embodiments, the composition is administered intravaginal in the form of 1 ovule, each containing a concentration of CBD: 100 mg, administered daily for 12 weeks. In some embodiments, the composition includes cannabinoids as active principles. In a further embodiment, the composition comprises cannabinoids ranging from 5% to 10%. In a preferred embodiment, the composition comprises CBD. In a further preferred embodiment, the composition comprises CBD 5%.

In another embodiment, the composition additionally contains lipid and antioxidant agents. In a further embodiment, the stabilizing and lipid agents range from 90%-95%, and the antioxidants from 0.07% to 0.10%. In a preferred embodiment, the composition comprises lipid agents such as medium-chain triglycerides (MCT) and cocoa butter and antioxidants such as vitamin E. In a further preferred embodiment, the composition comprises MCT at 32.5%, cocoa butter at 62.4%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for intravaginal administration and presented in ovules.

### Methods of Treatment for Menopause

In some embodiments, this product is presented in various pharmaceutical forms, including capsules for oral administration and ovules for intravaginal administration, specifically targeting a range of symptoms associated with menopause. In some embodiments, the method for treating menopause syndrome in a human individual comprises administering an effective concentration of the composition, wherein the active principle comprises a cannabinoid, flavonoids, polyphenols and the stabilizing solubilizing agent.

In some embodiments, this product is presented as capsules for oral administration, specifically targeting mood and sleep disorders, vasomotor symptoms such as hot flushes, bone loss and osteopenia, and reducing cardiovascular disease (CVD) risk associated with menopause. In some embodiments, this product is presented as ovules for intravaginal administration, specifically targeting symptoms of vaginal dryness and discomfort associated with genitourinary syndrome of menopause (GSM).

In further embodiments, this composition acts through a hypothalamic-pituitary-gonadal mechanism which benefits hormonal production and provides relief for the symptoms of menopause. It targets endocannabinoid receptors at the brain and gonadal levels, regulating the synthesis of sexual hormones such as estrogen and progesterone. The composition includes active phytocannabinoid compounds, flavonoids, and polyphenols.

In one embodiment, the pharmaceutical composition contains a combination of compounds including CBD (cannabidiol), CBG (cannabigerol), and CBN (cannabinol), which are selected for their potential therapeutic benefits in addressing menopause symptoms. In some embodiments, these cannabinoids are complemented by the inclusion of isoflavones, myricetin, quercetin, and resveratrol, which contribute additional anti-inflammatory, antioxidant, and bone-protective properties.

In one embodiment, the composition may be beneficial for treating menopause-related symptoms, such as vasomotor symptoms (VMS) including hot flashes and night sweats, genitourinary syndrome of menopause (GSM) characterized by vaginal dryness and discomfort, as well as bone loss, mood disturbances, and sleep disorders. Additionally, the composition may address associated risks like cardiovascular disease (CVD), providing a comprehensive approach to managing both the symptoms and long-term health impacts of menopause.

In one embodiment, the method describes a cannabinoid-based composition to alleviate menopausal vasomotor symptoms, such as hot flashes and night sweats. The composition may include cannabinoids like CBD and isoflavones to stabilize thermoregulation and reduce the frequency and intensity of these symptoms. In one embodiment, the method focuses on treating GSM symptoms, including vaginal dryness, irritation, and dyspareunia, through a cannabinoid-based composition. The composition is designed for oral and intravaginal applications, aiming to restore vaginal health and alleviate discomfort by targeting local inflammation and improving lubrication.

In one embodiment, the cannabinoid composition is intended to prevent bone loss and reduce osteoporosis risk in menopausal women. It may include CBD and bone-protective agents like myricetin or resveratrol to inhibit bone resorption and support bone density, counteracting the decline in bone health associated with estrogen deficiency.

In one embodiment addresses mood disturbances, anxiety, depression and sleep disorders during menopause by administering a cannabinoid-based composition. This composition modulates the endocannabinoid system to influence serotonin and dopamine levels, potentially enhanced by natural agents like quercetin to improve mood and promote restful sleep.

In one embodiment uses a cannabinoid composition to reduce increased cardiovascular risk during menopause. The combination of cannabinoids with polyphenols such as resveratrol and flavonoids like quercetin aims to improve endothelial function, reduce inflammation, and improve lipid profiles, ultimately lowering plasma triglycerides and cholesterol levels to mitigate cardiovascular disease risk in postmenopausal women.

In one embodiment, the patient may be female, specifically targeting those experiencing menopausal symptoms, including women in the perimenopausal phase and those who are postmenopausal, with the age range for treatment generally starting around 45 years and extending to over 65 years old. This range accounts for the natural onset of menopause, which typically occurs around age 49 and recognizes the broad spectrum of women affected by menopausal symptoms.

In one embodiment, the composition comprising nanoplatforms carrying phytocannabinoids is presented in a capsule and administered orally, with a treatment duration of ten to fourteen weeks based on symptom-based monitoring. In one embodiment, the method involves administering an effective concentration of the active principle within the range of 486 mg-594 mg per day. In one embodiment, the method involves administering a single capsule containing 540 mg of the active ingredients daily for 12 weeks.

In one embodiment, the composition comprising nanoplatforms carrying phytocannabinoids is presented in an ovule and intravaginally administered with a treatment duration of ten to fourteen weeks based on symptom-based monitoring. In one embodiment, the method involves administering an effective concentration of the active principle within the range of 90 mg-110 mg per day. In one embodiment, the method involves administering a single ovule containing 100 mg of the active ingredients daily for 12 weeks.

In further embodiments, the treatment method may vary based on the dosing regimen and schedule, which are critical for balancing efficacy with tolerability. For example, in further embodiments, a single-dose treatment method may be used where a high concentration of phytocannabinoids or other active ingredients is administered to provide rapid relief from severe menopausal symptoms, such as intense hot flashes or night sweats. This initial phase could be followed by a maintenance phase with lower doses to manage symptoms over time or combined with alternative therapies for ongoing symptom control.

In other embodiments, the treatment method may involve cyclical dosing, where the therapeutic agents are administered in cycles with defined treatment periods followed by rest intervals. This approach allows the body to adjust and recover, potentially reducing any side effects or cumulative stress, making it particularly suitable for long-term management of menopausal symptoms like mood disturbances or sleep disorders.

In further embodiments, continuous or prolonged dosing for cases where sustained suppression of vasovagal symptoms associated with menopause is desired may involve the continuous or prolonged administration of therapeutic agents through slow-release compositions, such as vaginal tablets, or through the use of oral compositions taken daily for extended periods.

In further embodiments, symptom-based monitoring may include periodic assessments, such as hormone level tests, bone density scans, or cardiovascular evaluations, to monitor the response to menopause treatment. This monitoring may guide the continuation, intensification, or modification of the treatment regimen based on changes in symptom severity, such as improvements in vasomotor symptoms (VMS), bone density, mood, or cardiovascular health.

In further embodiments, adaptive treatment plans for menopause may be designed to adjust the dose or suggest combination of therapies in response to changes in the patients symptoms or overall health. This approach ensures personalized care by modifying the treatment regimen based on improvements or worsening in symptoms like vasomotor symptoms (VMS), bone density, mood disturbances, or cardiovascular health, thereby optimizing the effectiveness and safety of the therapy throughout the treatment process.

### Method of Production of the Nanoplatforms/Nanoemulsion

In one embodiment, various delivery enhancements are employed to improve the efficacy and targeting of cannabinoid compositions. Methods may include liposomal encapsulation, nanoplatform as a nano emulsions and targeted delivery systems. In one embodiment, the production of nanoplatforms or nanoemulsions involves various methodologies that can be adapted to create the desired size, stability, and functionality of the final product. These methods can include multiple stages, specific excipients, and carefully controlled conditions such as temperature, pressure, and mixing techniques. Below, different variations in the production process are explored.

In one embodiment, nanoplatform compositions include nano particles with a mean diameter of less than 200 nm. In one embodiment, high-energy nano-emulsification methods are used to create stable nanoemulsions for effective delivery. In one embodiment, the choice of reactants, surfactants, and stabilizers is critical in the production process, influencing the stability, size, and functionality of the nanoplatforms or nanoemulsions. Various surfactants may be employed to stabilize the nanoemulsion, with the concentration and type varying according to the composition. Nonionic, cationic, or anionic surfactants can be utilized, and the hydrophilic-lipophilic balance (HLB) of the surfactant will affect the emulsification process and the size of the nanoplatform.

In one embodiment, the production method may also involve incorporating excipients that modify the surface of the nanoplatforms, enabling targeted delivery or enhanced interaction with biological systems. These excipients can include stabilizers, antioxidants, preservatives, as well as oil phase and aqueous vehicles. In one embodiment, polymers of lipids serve as the primary building blocks for the nanoplatforms. The production method may vary the type and molecular weight of the polymers or the chain length and saturation of the lipids, influencing the mechanical stability and release properties of the nanoplatform.

In one embodiment, the conditions under which the nanoplatforms or nanoemulsions are produced significantly impact the final properties, with adjustments made for different compositions. Temperature control is essential in various production methods, where the temperature is carefully regulated to affect the formation and stability of the nanoemulsion or nanoplatforms. Higher temperatures can decrease the viscosity of the mixture, aiding the emulsification process while risking the degradation of sensitive components.

In one embodiment, variations in pressure can be applied for high-pressure techniques, such as homogenization, to control the size and distribution of nanoplatform. Higher pressures may yield smaller particle sizes but may increase energy costs and the risk of degrading sensitive compounds. The number of pressure cycles can also be adjusted to ensure uniform nanoplatform formation.

In one embodiment, the production method may include mixing the reactants under high shear or vortex conditions to guarantee even distribution of particles and prevent agglomeration. The mixing speed and duration can be optimized to achieve the desired particle size and homogeneity, with different types of mixers, such as rotor-stator mixers, employed based on the composition requirements.

In one embodiment, various production techniques may be employed to create nanoemulsions, depending on the desired characteristics and application. These methods may include high-pressure homogenization, ultrasonication, microfluidization, or solvent evaporation/precipitation techniques.

In one embodiment, the production of nanoemulsions may involve high-pressure homogenization. This method utilizes high shear forces to break down larger particles into nanoscale sizes. The pressure and number of cycles can be adjusted to achieve the desired particle size and stability. In one embodiment, ultrasonication may be employed as the production method, where ultrasonic waves create cavitation to disrupt droplets or particles into nanoscale sizes. The sonication time, power, and frequency may be varied to control the properties of the resulting nanoemulsions.

In one embodiment, microfluidization may be used in the production process. The reactants are passed through microchannels at high velocities, creating uniform and stable nanostructures. The flow rate, pressure, and channel design can be modified to control the particle size and distribution of the nanoemulsions.

In these embodiments, nanoemulsions can be generated through solvent evaporation or precipitation techniques. An organic solvent containing the active ingredients is emulsified in an aqueous phase, followed by the evaporation of the solvent, which leads to the formation of nanoplatform. The choice of solvent, emulsifying agents, and evaporation conditions (such as temperature and pressure) can be adjusted to achieve the desired characteristics.

In one embodiment, the cannabinoid composition is administered daily over a period of ten to fourteen weeks using nanoplatforms to enhance the bioavailability, safety and efficacy of the compounds.

### Capsule Production Process

In one embodiment, the method of production may involve multiple stages, each designed to refine the properties of the nanoplatforms or nanoemulsions. In one embodiment, the preparation of the oil phase occurs in a suitable container, where one or more oils, such as medium or long-chain triglycerides, essential oils, or bioactive lipids, are combined with lipophilic excipients and any liposoluble active ingredients.

In one embodiment, the aqueous phase is prepared simultaneously in another container. This phase consists in purified water along with hydrophilic surfactants and co-emulsifying agents. In one embodiment, preliminary emulsification is carried out after both phases have reached the appropriate temperature, achieved by combining the oil phase and the aqueous phase to form the preliminary emulsion.

In one embodiment, the droplet size of the preliminary emulsion is reduced to a nanometric range through the application of high-energy homogenization techniques, which is crucial for obtaining the desired nanoemulsion. In one embodiment, after homogenization, the resulting nanoemulsion undergoes a controlled cooling process to ensure its stability. In one embodiment, the nanoemulsion is filled into a capsule with rigorous controls during the process.

### Ovule Production Process

In one embodiment of the disclosure, a method for manufacturing ovules involves the preparation of active ingredients and excipients in predetermined amounts. The components are weighed using precision scales to ensure that the quantities of active ingredients and excipients remain within the appropriate ranges, guaranteeing the uniformity and effectiveness of the final product.

In one embodiment, the disclosure contemplates a controlled melting process of the lipid base of the ovules, which may include cocoa butter or other lipids, which consists of heating the cocoa butter in a bain-marie at controlled temperature between 40-50° C. This melting is carried out gradually to avoid thermal degradation of the components, ensuring that the cocoa butter maintains its emollient and stabilizing properties.

In other embodiments, the active ingredients, such as cannabinoids, vitamins, or other active agents, are added to the melted lipid base. These components are dissolved or uniformly dispersed through constant stirring, ensuring that the active ingredient is fully distributed within the lipid matrix, which allows for homogeneous and controlled release upon administration.

In one embodiment, medium chain triglycerides are used to improve the solubility of CBD and increase the bioavailability of the active ingredient in the lipid matrix. The mixture of the lipid base and active ingredients is homogenized to ensure even distribution of the active ingredient throughout the ovule. This is achieved by continuous stirring using a suitable mixer. Homogenization is critical to ensure that each ovule contains a consistent amount of active ingredients. In another embodiment, the homogeneous mixture is poured into pre-sterilized molds. These molds are designed to give the ovule the proper shape and size for administration. The pouring process is performed precisely to ensure that each mold receives an exact amount of the mixture to maintain consistent dosage. In one embodiment, the disclosure includes pouring the homogeneous mixture into molds designed to shape the ovules. The liquid mixture is precisely distributed in the molds, ensuring that each ovule has a correct weight.

In one embodiment, the disclosure contemplates a controlled cooling process for the solidification of the ovules. Once the mixture has been poured into the molds, these are allowed to cool to room temperature or to a temperature between 4-8° C. This cooling process ensures a uniform crystallization of the cocoa butter, which improves the physical stability of the ovule and the uniformity in the release of the CBD. In one embodiment, the solidified ovules are carefully removed from the molds to avoid damaging their structure. This process can be carried out manually or through an automated system that ensures the physical integrity of the ovule. In one embodiment, the cannabinoid composition is administered daily over a period of ten to fourteen weeks, using ovules to enhance the bioavailability, safety and efficacy of the compounds.

### EXAMPLES

### Pharmaceutical Compositions

The current example discloses a therapeutic agent aimed at treating menopause. This agent comprises a unique composition that utilizes nanoplatform containing cannabinoids, such as CBD, CBG and CBN. The composition is defined by designated percentage ranges of each to ensure effective administration. The example describes a substance designed to treat menopause in patients. This substance comprises a composition that utilizes nanoplatform containing cannabinoids, such as CBD, CBG and CBN. Each compound percentage range for administration is outlined to optimize therapeutic effects. Particular amounts of cannabinoids: 100 mg of CBD, 5 mg of CBG, and 2.5 mg of CBN per capsule. Particular amounts of cannabinoids: 100 mg of CBD per ovule.

### Capsules:

### Composition 1:940 mg Per Capsule

| Component | Quantity (mg) | Percentage (%) |
|---|---|---|
| CBD | 100 | 10.64 |
| CBG | 5 | 0.53 |
| CBN | 2.5 | 0.27 |
| Resveratrol | 70 | 7.45 |
| Quercetin | 250 | 26.60 |
| Myricetin | 50 | 5.32 |
| Soy Isoflavones | 60 | 6.38 |
| Medium chain triglycerides | 108 | 11.49 |
| Sorbitan 80 (Span 80) | 20 | 2.13 |
| Vitamin E | 0.94 | 0.10 |
| Polysorbate 80 (Tween 80) | 20 | 2.13 |
| Polyethylene glycol 400 | 25 | 2.66 |
| Methylparaben | 1.692 | 0.18 |
| Propylparaben | 0.188 | 0.02 |
| Ethanol | 75.2 | 8.00 |
| Purified Water | 151.48 | 16.11 |

### Composition 2:1200 mg Per Capsule

| Component | Quantity (mg) | Percentage (%) |
|---|---|---|
| CBD | 100 | 8.33 |
| CBG | 5 | 0.42 |
| CBN | 2.5 | 0.21 |
| Resveratrol | 70 | 5.83 |
| Quercetin | 250 | 20.83 |
| Myricetin | 50 | 4.17 |
| Soy Isoflavones | 60 | 5.00 |
| Medium chain triglycerides | 240.00 | 20.00 |
| Sorbitan 80 (Span 80) | 50.00 | 4.17 |
| Vitamin E | 1.20 | 0.10 |
| Polysorbate 80 (Tween 80) | 50.00 | 4.17 |
| Polyethylene glycol 400 | 12.00 | 1.00 |
| Methylparaben | 2.16 | 0.18 |
| Propylparaben | 0.24 | 0.02 |
| Ethanol | 120.00 | 10.00 |
| Purified Water | 276.90 | 23.08 |

### Ovules:

### Composition 3:1000 mg Per Ovule

| Component | Quantity (mg) | Percentage (%) |
|---|---|---|
| CBD | 100 | 10.00 |
| Vitamin E | 1 | 0.10 |
| Medium chain triglycerides | 299 | 29.90 |
| Cocoa butter | 600 | 60.00 |

### Composition 4:2000 mg Per Ovule

| Component | Quantity (mg) | Percentage (%) |
|---|---|---|
| CBD | 100 | 5.00 |
| Vitamin E | 2 | 0.10 |
| Medium chain triglycerides | 650 | 32.50 |
| Cocoa butter | 1248 | 62.40 |

### Nanoemulsion Process:

### Example 1

### Oil Phase:

A quantity of 11.49 grams of medium-chain triglycerides (MCT) is added to a suitable container. Subsequently, 2.13 grams of Sorbitan 80 (Span 80) and 0.1 grams of Vitamin E are incorporated. Immediately, 10.64 grams of CBD, 0.53 grams of CBG and 0.27 grams of CBN is added. The mixture is stirred at a speed of 500 revolutions per minute (rpm) until all components are fully integrated. The oil phase is then heated to a temperature between 40° C. and 45° C.

### Aqueous Phase:

A quantity of 8 grams of alcohol is added to a suitable container. Subsequently, 7.45 grams of resveratrol are incorporated. Immediately, 5.32 grams of myricetin are added. The mixture is stirred at a speed of 500 revolutions per minute (rpm) until all components are fully integrated. The solution is then reserved for further use. Heat 16.11 grams of purified water to 90° C. and dissolve 0.18 grams of methylparaben and 0.02 grams of propylparaben. Cool the solution to a temperature between 40° C. and 45° C. Add 2.66 grams of polyethylene glycol 400 (PEG 400) and 2.13 grams of polysorbate 80 (Tween 80), and stir until all ingredients are completely incorporated. Add 6.38 grams of Soy Isoflavones and continue stirring until all components are fully integrated. Then, add 26.60 grams of quercetin and stir until fully dissolved. Incorporate the reserved solution of ethanol-resveratrol-myricetin and stir the resulting mixture at a speed of 500 rpm until all ingredients are fully integrated. Maintain the temperature between 40° C. and 45° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-energy ultrasonic homogenizer is used. The sonication process is carried out in 10-second sonication cycles followed by 10 seconds of rest, for a total period of 15 to 20 minutes. During the entire sonication process, the temperature of the emulsion is maintained at 45° C.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into soft gelatin capsules.

### Example 2

Oil phase: A quantity of 12.50 grams of medium-chain triglycerides (MCT) is added to a suitable container. Subsequently, 4.17 grams of Sorbitan 80 (Span 80) and 0.1 grams of Vitamin E are incorporated. Immediately, 8 grams of CBD, 0.42 grams of CBg, and 0.21 grams of CBN is added. The mixture is stirred at a speed of 350-700 revolutions per minute (rpm) until all components are fully integrated. The oil phase is then heated to a temperature between 60° C. and 70° C.

Aqueous phase: A quantity of 10 grams of alcohol is added to a suitable container. Subsequently, 5.83 grams of resveratrol are incorporated. Immediately, 4.17 grams of myricetin are added. The mixture is stirred at a speed of 350-700 revolutions per minute (rpm) until all components are fully integrated. The solution is then reserved for further use. Heat 23.08 grams of purified water to 90° C. and dissolve 0.18 grams of methylparaben and 0.02 grams of propylparaben. Cool the solution to a temperature between 60° C. and 70° C. Add 1.00 grams of polyethylene glycol 400 (PEG 400) and 4.17 grams of polysorbate 80 (Tween 80), and stir until all ingredients are completely incorporated. Add 6.38 grams of Soy Isoflavones and continue stirring until all components are fully integrated. Then, add 20.83 grams of quercetin and stir until fully dissolved. Incorporate the reserved solution of ethanol-resveratrol-myricetin and stir the resulting mixture at a speed of 350-700 rpm until all ingredients are fully integrated. Maintain the temperature between 60° C. and 70° C.

Formation of the preliminary emulsion: Once both the oil and aqueous phases have reached the established temperature, the oil phase is slowly and gradually added to the aqueous phase, under constant stirring. The mixture is stirred for a period of 30 minutes to ensure the formation of a homogeneous preliminary emulsion.

Formation of the nanoemulsion: To reduce the droplet size in the preliminary emulsion to a range of 50 to 200 nanometers (nm), a high-pressure homogenizer is used. The homogenization process is carried out by applying pressures between 1000 and 1500 bar, over 3 to 5 cycles of homogenization. During each cycle, the emulsion is forced through a narrow valve at high speed, generating shear forces that reduce the droplet size. It is crucial to monitor the temperature of the emulsion throughout the process, keeping it between 60° C. and 70° C., using a cooling system if necessary, to preserve the stability of the active ingredients.

Cooling of the nanoemulsion: After the sonication process is completed, the resulting nanoemulsion is cooled until it reaches a temperature below 30° C., while stirring at a low speed between 30 and 50 rpm, to ensure uniform droplet distribution during cooling.

Capsule filling: Once the nanoemulsion has reached the appropriate temperature, the filling of the capsules is carried out. The nanoemulsion is introduced into vegetarian capsules.

### Ovule Manufacture Process:

### Example 3

Melting the Base: 60 grams of cocoa butter is heated in a double boiler at a temperature of approximately 40-50° C. until it is completely melted, ensuring that it is not overheated to avoid degradation of its properties.

Incorporation of the Active Ingredients: Once the cocoa butter is melted, 10.0 grams of CBD and 0.10 grams of vitamin E are added, mixing well to ensure their complete dissolution in the oily base. Next, 29.90 grams of medium chain triglycerides are incorporated, which act as carriers and improve the bioavailability of the CBD.

Homogenization: The mixture is stirred for several minutes to ensure that all the components are well dispersed and that the mixture is homogeneous. The temperature is maintained in the range of 40-50° C.

Molding the Ovules: Silicone or metal molds are prepared, which are cleaned and disinfected. The liquid mixture is carefully poured into the molds, filling them to the desired level. It is left to cool at room temperature or in the fridge to allow the ovules to solidify.

Unmoulding: Once the mixture has solidified, the ovules are carefully unmoulded. The quality of the ovules is checked visually to ensure that they do not have any visible defects.

### Example 4

Incorporation of the Active Ingredients: 32.50 grams medium chain triglycerides are heated in a double boiler at a temperature of approximately 40-50° C., then 5.0 grams of CBD and 0.10 grams of vitamin E are added, mixing well to ensure their complete dissolution in the oily base.

Melting the Base: Once the CBD and Vitamin E are dissolved, 62.40 grams of cocoa butter is added, the temperature continues at 40-50° C. until it is completely melted.

Homogenization: The mixture is stirred for several minutes to ensure that all the components are well dispersed and that the mixture is homogeneous. The temperature is maintained in the range of 40-50° C.

Molding the Ovules: Silicone or metal molds are prepared, which are cleaned and disinfected. The liquid mixture is carefully poured into the molds, filling them to the desired level. It is left to cool at room temperature or in the fridge to allow the ovules to solidify.

Unmoulding: Once the mixture has solidified, the ovules are carefully unmoulded. The quality of the ovules is checked visually to ensure that they do not have any visible defects.

Treatment kit: The purpose of the kit is to provide systemic and localized relief of menopause symptoms, offering a natural approach to improving the quality of life during the menopausal period transition. The example comprises a kit designed for the comprehensive treatment of menopause symptoms, consisting of two products: capsules and ovules:

Product 1—capsules: is a composition comprising cannabinoids, flavonoids, and polyphenols, specifically: CBD: 100 mg; CBG: 5 mg; CBN: 2.5 mg; Resveratrol: 70 mg; Quercetin: 250 mg; Myricetin: 50 mg; Isoflavones: 60 mg.

The composition is orally administered in the form of 2 capsules, each containing a concentration of: CBD: 100 mg, CBG: 5 mg, CBN: 2.5 mg, Resveratrol: 70 mg, Quercetin: 250 mg, Myricetin: 50 mg, Isoflavones: 60 mg. This treatment will be taken daily, 1 capsule in the morning and 1 capsule at night for 12 weeks. The capsules are developed using a nanoemulsion technique to improve the bioavailability and efficacy of the active compounds.

In one non-limiting embodiment, this composition for menopause care contains the following active ingredients per dosage: CBD—100 mg; CBG—5 mg; CBN—2.5 mg. In one preferred embodiment, the composition comprises the following concentrations: CBD at 10.64%, CBG at 0.53%, and CBN at 0.27%.

In a further embodiment, the flavonoids in the composition include isoflavones, quercetin, and myricetin, while the polyphenols include resveratrol. In one embodiment, the composition includes resveratrol at a concentration ranging from 5.50% to 7.50% per capsule. In another embodiment, the composition includes quercetin at a concentration ranging from 20% to 27% per capsule.

In a further embodiment, the composition includes myricetin at a concentration ranging from 4% to 6% per capsule. In an additional embodiment, the composition includes isoflavonoids at a concentration ranging from 5% to 7% per capsule. In some embodiments, the composition includes cannabinoids, flavonoids, and polyphenols as active principles. In a further embodiment, the composition comprises cannabinoids ranging from 8% to 13% and flavonoids ranging from 35% to 46% per capsule. In a preferred embodiment, the composition comprises cannabinoids such as CBD, CBG, and CBN, and flavonoids and polyphenols such as isoflavones, myricetin, quercetin, and resveratrol. In a further preferred embodiment, the composition comprises CBD 10.64%, CBG 0.53%, CBN 0.27%, isoflavones 6.38%, myricetin 5.32%, quercetin 26.6%, and resveratrol 7.45%.

In another embodiment, the composition additionally contains stabilizing agents and antioxidant agents. In a further embodiment, the stabilizing agents range from 6.91% to 9.33%, and the antioxidants from 0.07% to 0.10%. In a preferred embodiment, the composition comprises stabilizing agents such as PEG-400 and Tween 80, and antioxidants such as vitamin E. In a further preferred embodiment, the composition comprises PEG-400 at 2.13%, Tween at 2.66%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for oral administration and presented in capsules.

Product 2—ovules: is formulated to address vaginal dryness and contains: CBD: 100 mg.

In some embodiments, the composition is administered intravaginal in the form of 1 ovule, each containing a concentration of: CBD: 100 mg, administered daily for 12 weeks. In some embodiments, the composition includes cannabinoids as active principles. In a further embodiment, the composition comprises cannabinoids ranging from 5% to 10%. In a preferred embodiment, the composition comprises CBD. In a further preferred embodiment, the composition comprises CBD 5%.

In another embodiment, the composition additionally contains lipid and antioxidant agents. In a further embodiment, the stabilizing and lipid agents range from 90%-95%, and the antioxidants from 0.07% to 0.10%. In a preferred embodiment, the composition comprises lipid agents such as medium-chain triglycerides (MCT) and cocoa butter and antioxidants such as vitamin E. In a further preferred embodiment, the composition comprises MCT at 32.5%, cocoa butter at 62.4%, and vitamin E at 0.1%.

In some embodiments, this pharmaceutical composition is designed for multiple routes of administration and pharmaceutical forms. In a further embodiment, the routes of administration may include oral, sublingual, topic, inhalation, parenteral, and intravaginal, while the pharmaceutical forms include tablets, capsules, oral drops, syrups, suspensions, powders for oral reconstitution, elixirs, creams, emulsions, oral solutions, gummies, caramels, sprays, oral topicals, mouth rinses, sublingual tablets, sublingual films, ovules, suppositories, injectable solutions, and injectable suspensions. In a preferred embodiment, this pharmaceutical composition is intended for intravaginal administration and presented in ovules.

### Therapeutic Kit for Menopause Symptom Management

### Example 1: Menopause Symptom Treatment Kit

This example illustrates the use of a therapeutic kit specifically designed for the comprehensive management of menopause symptoms. The kit includes two primary products: oral capsules and intravaginal ovules, each formulated to target a range of menopausal symptoms, including vasomotor disturbances (hot flashes), genitourinary syndrome of menopause (GSM), mood swings, sleep disturbances, and cardiovascular health.

Oral Capsules: Composition: Each oral capsule contains the following active ingredients: Cannabidiol (CBD): 100 mg; Cannabigerol (CBG): 5 mg; Cannabinol (CBN): 2.5 mg; Resveratrol: 70 mg; Quercetin: 250 mg; Myricetin: 50 mg; Soy Isoflavones: 60 mg.

The active ingredients are encapsulated using nanoemulsion technology to improve bioavailability, ensuring effective absorption in the body. The cannabinoids interact with the endocannabinoid system (ECS) to help regulate mood, pain, and thermoregulation, addressing vasomotor symptoms such as hot flashes, while the polyphenols and flavonoids offer antioxidant and cardioprotective benefits.

Other Ingredients: Medium-chain triglycerides (MCT): 108 mg; Sorbitan 80 (Span 80): 20 mg; Vitamin E: 0.94 mg; Polysorbate 80 (Tween 80): 20 mg; Polyethylene glycol 400 (PEG 400): 25 mg; Methylparaben: 1.692 mg; Propylparaben: 0.188 mg; Ethanol: 75.2 mg; Purified Water: 151.48 mg.

### Dosage and Administration:

The oral capsules are to be taken twice daily-one in the morning and one in the evening. The recommended course of treatment is 12 weeks to effectively manage the range of menopausal symptoms, including hot flashes, mood disturbances, and sleep disorders, while providing long-term cardiovascular benefits.

Mechanism of Action: CBD, CBG, and CBN modulate the endocannabinoid system (ECS) to alleviate mood swings and improve sleep patterns, while also reducing pain and inflammation commonly associated with menopausal symptoms. Resveratrol, quercetin, and myricetin provide antioxidant and cardiovascular support, helping to reduce the risk of cardiovascular disease in postmenopausal women by enhancing endothelial function and improving lipid profiles. Soy Isoflavones mimic mild estrogenic effects, aiding in bone density preservation and alleviating vasomotor symptoms like hot flashes.

Intravaginal Ovules: Composition: Each ovule contains: Cannabidiol (CBD): 100 mg; Medium-chain triglycerides (MCT): 299 mg; Cocoa Butter: 600 mg; Vitamin E: 1 mg.

The lipid-based formulation utilizes cocoa butter and MCT to ensure that the CBD is effectively delivered to the vaginal tissues. This composition provides localized relief for genitourinary symptoms of menopause (GSM), such as vaginal dryness, irritation, and discomfort during intercourse.

### Dosage and Administration:

The ovules are administered intravaginally once daily, preferably at night to allow for optimal absorption and prolonged contact with vaginal tissues. The recommended duration of treatment is 12 weeks.

### Mechanism of Action:

CBD interacts with local endocannabinoid receptors (CB1 and CB2) to reduce inflammation and pain, promoting lubrication and improving tissue elasticity. The lipid base of cocoa butter and MCT provides a soothing and moisturizing effect, addressing vaginal dryness and irritation, while also ensuring the efficient absorption of CBD.

### Treatment Protocol:

Patients undergoing treatment with this menopause symptom management kit will follow the below protocol: Morning Regimen: Take one oral capsule with water, preferably after a meal. Evening Regimen: Take one oral capsule after dinner. Insert one intravaginal ovule before bed, ensuring a comfortable position for insertion. The dual approach—combining oral systemic treatment with localized intravaginal treatment—ensures comprehensive relief from both systemic and localized menopausal symptoms. The combination of cannabinoids, flavonoids, and polyphenols targets the physiological and psychological challenges associated with menopause, offering a natural alternative to conventional hormone therapy (HT).

### Benefits of the Kit:

Multidimensional Symptom Relief: The oral capsules target systemic symptoms, such as hot flashes, mood swings, and cardiovascular risks, while the intravaginal ovules provide localized relief from GSM, addressing vaginal dryness and irritation.

High Bioavailability: The use of nanoplatform technology ensures that the cannabinoids, flavonoids, and polyphenols in the capsules are efficiently absorbed into the bloodstream, offering improved therapeutic outcomes with lower doses.

Cardiovascular and Bone Health: The combination of resveratrol, quercetin, and soy isoflavones provides additional health benefits, helping to improve endothelial function and preserve bone density, reducing the risks of cardiovascular disease and osteoporosis.

Localized and Systemic Effectiveness: By combining oral and intravaginal delivery systems, the kit ensures that both systemic and localized symptoms of menopause are effectively managed, leading to improved quality of life for patients.

Natural and Safe Alternative: The use of cannabinoids and phytoestrogens offers a safer and natural alternative to traditional hormone therapy, minimizing the risks associated with synthetic hormones, such as venous thromboembolism (VTE) and breast cancer.

This example outlines the use of a therapeutic kit containing oral capsules and intravaginal ovules designed for the comprehensive management of menopausal symptoms. The combination of cannabinoids, flavonoids, polyphenols, and isoflavones ensures a holistic approach to managing both systemic and localized symptoms, offering a safe and effective treatment for women undergoing the menopausal transition. The use of nanoemulsion technology enhances the bioavailability of the active ingredients, making this kit a cutting-edge solution for menopause management.

1. A composition for treatment of menopause syndrome, comprising:
a) an active principle comprising at least one cannabinoid; and
b) a lipid agent.

2. The composition according to claim 1 wherein further comprises a second active principle comprising a flavonoid, an isoflavone, a polyphenol, or mixtures thereof.

3. The composition according to claim 1, wherein the composition is an oral form or ovule form.

4. The composition according to claim 1, wherein the cannabinoid is present in the range of 5% to 20%.

5. The composition according to claim 4, wherein the cannabinoid comprises one or more of the following: cannabidiol (CBD), cannabigerol (CBG), cannabichromene (CBN) or mixtures thereof.

6. The composition according to claim 5, wherein CBD is present in the range of 5%-10%.

7. The composition according to claim 5, wherein CBG is present in the range of 0.43%-0.52%, and CBN is present in the range of 0.20%-0.27%.

8. The composition according to claim 1, wherein further comprises a stabilizing solubilizing agent, which is present in the range of 30%-33%.

9. The composition according to claim 1, wherein CBD is present in a concentration 5%-10% and is present into ovule form.

10. The composition according to claim 1, wherein CBD is present in a concentration in the range of 8%-11%, CBG 0.4%-0.6%, CBN 0.15%-0.30%; and is present in an oral form.

11. The composition according to claim 1, wherein isoflavone is present in a concentration of 5%-10% and is soy isoflavones; polyphenol is present in a concentration of 5%-8% and is resveratrol; and flavonoid is present in a concentration of 4%-27% and are Quercetin, Myricetin and combinations thereof.

12. A method for treating menopause syndrome in a human individual, comprising: administering an effective concentration of the composition of claim 1.

13. The method according to claim 12, wherein the human individual is selected for treatment based on being diagnosed with menopause.

14. The method according to claim 12, wherein the individual is selected for treatment of: perimenopause, menopause and postmenopause.

15. The method of claim 13 wherein further comprises identifying individuals using: Medical history, Physical examination, Blood tests, Symptom diary or gynecological test.

16. The method of claim 13, wherein the individual is identified by having alterations in serum biomarkers, including increase in FSH levels and a decrease in estrogen levels.

17. The method according to claim 12, wherein a capsule comprising CBD: 100 mg, CBG: 5 mg, CBN: 2.5 mg, Resveratrol: 70 mg, Quercetin: 250 mg, Myricetin: 50 mg and Isoflavones: 60 mg is administered as a single capsule for a period of 12 weeks per day.

18. The method of claim 1, wherein an ovule comprising CBD: 100 mg is administered for a period of 12 weeks per day.

19. The method according to claim 12, wherein the composition acts as a monotherapy for treating menopause.

20. The method of claim 12, wherein the treatment composition alleviates: vasomotor symptoms (VMS), genitourinary syndrome of menopause (GSM), bone loss, joint pain, mood disturbances, and sleep disorders, while also addressing associated risks like cardiovascular disease (CVD).
